(19) United States
(12) Patent Application Publication
(10) Pub. No.: US 2015/0072391 A1
(43) Pub. Date: Mar. 12, 2015

(54) ETHANOL PRODUCTION IN ENGINEERED YEAST

(71) Applicants: Massachusetts Institute of Technology, Cambridge, MA (US); Whitehead Institute for Biomedical Research, Cambridge, MA (US)

(72) Inventors: Felix Lam, Cambridge, MA (US); Gerald Fink, Chestnut Hill, MA (US); Gregory Stephanopoulos, Winchester, MA (US)

(73) Assignees: Massachusetts Institute of Technology, Cambridge, MA (US); Whitehead Institute for Biomedical Research, Cambridge, MA (US)

(21) Appl. No.: 14/479,118

(22) Filed: Sep. 5, 2014

Related U.S. Application Data
(60) Provisional application No. 61/874,793, filed on Sep. 6, 2013.

(51) Int. Cl. C12P 7/10; C12N 15/81

(52) U.S. Cl. CPC C12P 7/10; C12N 15/81
USPC 435/165; 435/255.1; 435/254.2; 435/471

(57) ABSTRACT

The present disclosure provides, in various aspects, engineered alcohol tolerant yeast and methods of producing high concentrations of ethanol.

### RELATED APPLICATION

This application claims the benefit under 35 U.S.C. §119(e) of U.S. provisional application No. 61/874,793, filed Sep. 6, 2013, which is incorporated by reference herein in its entirety.

### FEDERALLY SPONSORED RESEARCH

This invention was made with government support under Grant No. DK075850 awarded by the National Institutes of Health and under Grant No. DE-FC36-07GO17058 awarded by the Department of Energy. The government has certain rights in the invention.

### BACKGROUND OF THE INVENTION

The increased use of renewable transportation fuels such as bioethanol is one of the most widely accepted strategies to combat global climate change¹. However, the toxicity of ethanol and other alcohols to the industrial production organism, Saccharomyces cerevisiae, is a primary factor limiting greater output. The high cell density (“pitch”) and very high sugar (“gravity”) conditions of large-scale fermentation produce preternaturally high concentrations of ethanol that lead to significant losses in cell viability and productivity^{2,3}. Ethanol tolerance is a complex phenotype with an elusive biological basis; genetic analysis has shown that no single modification is capable of eliciting greater resistance⁴⁻⁷.

### SUMMARY OF THE INVENTION

Ethanol toxicity in yeast S. cerevisiae limits the production of biofuels globally, yet its biological underpinnings remain enigmatic. Surprisingly, the present disclosure shows that the basis of general alcohol tolerance is the upkeep of the opposing potassium and proton electromotive membrane gradients. Potassium supplementation and acidity reduction of culture medium physically strengthen these gradients, significantly increasing ethanol production in very high sugar and high cell density conditions mimicking industrial fermentation. Ethanol production per viable cell remains unchanged, and the enhancement in total output derives solely from elevated viability. Tolerance to ethanol can be controlled genetically, for example, via modulation of the cognate potassium (K⁺) and proton (H⁺) pumps; the artificially facilitated/increased import of K⁺ and export of H⁺ confer characteristics on laboratory strains that match or exceed those of industrial strains. Potassium supplementation and acidity reduction, furthermore, raise ethanol performance universally among a sampling of industrial and laboratory strains, including one engineered to ferment xylose. Moreover, these ionic adjustments increase resistance to isopropanol and isobutanol. The present disclosure reveals that alcohol tolerance, while amenable to genetic augmentation, is dominated by a major physicochemical component.

Thus, various aspects of the disclosure provide an alcohol tolerant yeast cell engineered to comprise a modified potassium transport gene encoding a polypeptide that increases cellular influx of potassium relative to an unmodified yeast cell and a modified proton transport gene encoding a polypeptide that increases the cellular efflux of protons relative to an unmodified yeast cell. In some embodiments, an alcohol tolerant yeast cell is further engineered to express an enzyme that converts aldehydes into their equivalent alcohols. The enzyme may be, for example, an alcohol dehydrogenase (e.g., obtained from Saccharomyces cerevisiae or Scheffersomyces stipitis), an aldehyde dehydrogenase (e.g., obtained from Saccharomyces cerevisiae or Escherichia coli), an aldehyde reductase (e.g., obtained from Saccharomyces cerevisiae), an oxidative stress activator (e.g., obtained from Saccharomyces cerevisiae), a catalase activated by YAP1 (e.g., obtained from Saccharomyces cerevisiae), a xylose reductase (e.g., obtained from Scheffersomyces stipitis) or a methylglyoxal reductase (e.g., obtained from Escherichia coli). In some embodiments, the enzyme is an alcohol dehydrogenase (e.g., obtained from Saccharomyces cerevisiae) such as ADH1, ADH2, ADH6, ADH7 or SFA1. In some embodiments, the enzyme is an aldehyde dehydrogenase (e.g., obtained from Saccharomyces cerevisiae) such as ALD4 or ALD5. In some embodiments, the enzyme is an aldehyde reductase (e.g., obtained from Saccharomyces cerevisiae) such as GRE3 or ARI1.

In some embodiments, the intracellular potassium in the engineered yeast cell is maintained at a concentration of about 100 mM to about 400 mM and the intracellular pH is maintained at about 5.5 to about 8.5. In some embodiments, the intracellular potassium is maintained at a concentration of about 200 mM to about 300 mM. In some embodiments, the intracellular pH in the engineered yeast cell is maintained at about 7.

In some embodiments, the alcohol tolerant yeast cell is tolerant to ethanol, isopropanol and/or isobutanol.

In some embodiments, the potassium transport gene comprises a deletion mutation. In some embodiments, the potassium transport gene is overexpressed. In some embodiments, the proton transport gene comprises a deletion mutation. In some embodiments, the proton transport gene is overexpressed. In some embodiments, the potassium transport gene is selected from TRK1, TRK2, PPZ1, PPZ2 and an HAL family member. In some embodiments, the proton transport gene is selected from PMA1, PMA2 and a VMA family member.

In some embodiments, the alcohol tolerant yeast cell comprises a modified sodium transport gene. In some embodiments, the modified sodium transport gene encodes a polypeptide that increases the cellular efflux of sodium relative to an unmodified yeast cell. In some embodiments, the modified sodium transport gene comprises a deletion mutation or is overexpressed. In some embodiments, the modified sodium transport gene is selected from NHA1 and an ENA family member.

In some embodiments, the alcohol tolerant yeast cell is an engineered ppz1Δ/ppz2Δ yeast cell that overexpresses PMA1.

In some embodiments, the unmodified yeast cell is a Saccharomyces cerevisiae cell. In some embodiments, the unmodified yeast cell is of an industrial yeast cell. In some embodiments, the unmodified yeast cell is a NCYC 479 (Sake) yeast cell. In some embodiments, the unmodified yeast cell is a PE-2 (Bioethanol) yeast cell (also referred to as JAY270). In some embodiments, the unmodified yeast cell is an ETHANOL RED® cell.

In some embodiments, the alcohol tolerant yeast cell has been previously modified to produce ethanol, isopropanol or isobutanol.

In some embodiments, the alcohol tolerant yeast cell expresses a cellulase and/or a hemicellulase.

Also provided herein is a method of producing alcohol, the method comprising culturing, in culture medium that comprises fermentable feedstock, any of the foregoing alcohol tolerant yeast cells, thereby producing alcohol. In some embodiments, the alcohol is ethanol, isopropanol or isobutanol.

In some embodiments, the fermentable feedstock is cellulosic feedstock. In some embodiments, the fermentable feedstock is fermentable sugar. In some embodiments, the fermentable sugar is glucose. In some embodiments, the fermentable sugar is xylose. In some embodiments, the concentration of the fermentable sugar is about 50 g/L to about 400 g/L.

In some embodiments, a plurality of the alcohol tolerant yeast cells is cultured at an OD₆₀₀ of about 15 to 50.

In some embodiments, at least 80 g/L to at least 150 g/L alcohol (e.g., ethanol) is produced. For example, in some embodiments, at least 80 g/L, at least 90 g/L or at least 100 g/L, at least 110 g/L, at least 120 g/L, at least 130 g/L, at least 140 g/L or at least 150 g/L of alcohol (e.g., ethanol) is produced. In some embodiments, at least 80 g/L to at least 150 g/L alcohol (e.g., ethanol) is produced over the course of 1 to 4 days (or at least 1 to 4 days) (e.g., 2 to 3 days), or more. For example, in some embodiments, at least 80 g/L, at least 90 g/L or at least 100 g/L, at least 110 g/L, at least 120 g/L, at least 130 g/L, at least 140 g/L or at least 150 g/L of alcohol (e.g., ethanol) is produced over the course of 1 to 4 days (or at least 1 to 4 days) (e.g., 2 to 3 days), or more.

In some embodiments, the culture medium further comprises a potassium salt, such as potassium phosphate monobasic (KH₂PO₄), potassium phosphate dibasic (K₂HPO₄) or potassium sulfate (K₂SO₄). Thus, in some embodiments, engineered yeast cells (e.g., alcohol tolerant yeast cells engineered to comprise a modified potassium transport gene encoding a polypeptide that increases cellular influx of potassium relative to an unmodified yeast cell and a modified proton transport gene encoding a polypeptide that increases the cellular efflux of protons relative to an unmodified yeast cell) are cultured in cell culture medium that comprises a potassium salt, such as potassium phosphate monobasic (KH₂PO₄), potassium phosphate dibasic (K₂HPO₄) or potassium sulfate (K₂SO₄). In some embodiments, the potassium salt is in an amount sufficient to produce at least 80 g/L to 150 g/L (e.g., least 80 g/L, at least 90 g/L or at least 100 g/L alcohol, at least 110 g/L, at least 120 g/L, at least 130 g/L, at least 140 g/L, at least 150 g/L, or more (e.g., ethanol). In some embodiments, culturing engineered yeast cells as provided herein in culture medium that comprises a potassium salt (e.g., KH₂PO₄, K₂HPO₄, K2SO₄) produces at least 150 g/L, or more, alcohol (e.g., at least 160 g/L or at least 170 g/L). In some embodiments, the potassium salt is in an amount sufficient to produce at least 80 g/L to 150 g/L (e.g., least 80 g/L, at least 90 g/L or at least 100 g/L alcohol, at least 110 g/L, at least 120 g/L, at least 130 g/L, at least 140 g/L, at least 150 g/L, or more (e.g., ethanol) over the course of 1 to 4 days (or at least 1 to 4 days) (e.g., 2 to 3 days), or more. In some embodiments, culturing engineered yeast cells as provided herein in culture medium that comprises a potassium salt (e.g., KH₂PO₄, K₂HPO₄, K2SO₄) produces at least 150 g/L, or more, alcohol (e.g., at least 160 g/L or at least 170 g/L) over the course of 1 to 4 days (or at least 1 to 4 days) (e.g., 2 to 3 days), or more. In some embodiments, the alcohol is ethanol, isopropanol or isobutanol.

Various other aspects of the disclosure provide a method of producing an alcohol tolerant yeast cell, the method comprising modifying in a yeast cell a potassium transport gene and a proton transport gene, thereby producing an alcohol tolerant yeast cell with an increased cellular influx of potassium and an increased cellular efflux of protons relative to an unmodified yeast cell.

In some embodiments, the method further comprises expressing (e.g., overexpressing) in the yeast cell an enzyme that converts aldehydes into their equivalent alcohols. The enzyme may be, for example, an alcohol dehydrogenase (e.g., obtained from Saccharomyces cerevisiae or Scheffersomyces stipitis), an aldehyde dehydrogenase (e.g., obtained from Saccharomyces cerevisiae or Escherichia coli), an aldehyde reductase (e.g., obtained from Saccharomyces cerevisiae), an oxidative stress activator (e.g., obtained from Saccharomyces cerevisiae), a catalase activated by YAP1 (e.g., obtained from Saccharomyces cerevisiae), a xylose reductase (e.g., obtained from Scheffersomyces stipitis) or a methylglyoxal reductase (e.g., obtained from Escherichia coli). In some embodiments, the enzyme is an alcohol dehydrogenase (e.g., obtained from Saccharomyces cerevisiae) such as ADH1, ADH2, ADH6, ADH7 or SFA1. In some embodiments, the enzyme is an aldehyde dehydrogenase (e.g., obtained from Saccharomyces cerevisiae) such as ALD4 or ALD5. In some embodiments, the enzyme is an aldehyde reductase (e.g., obtained from Saccharomyces cerevisiae) such as GRE3 or ARI1.

In some embodiments, the method further comprises culturing the alcohol tolerant yeast cell under conditions that produce ethanol, thereby producing ethanol.

In some embodiments, the potassium transport gene comprises a deletion mutation. In some embodiments, the potassium transport gene is overexpressed. In some embodiments, the proton transport gene comprises a deletion mutation. In some embodiments, the proton transport gene is overexpressed. In some embodiments, the potassium transport gene is selected from TRK1, TRK2, PPZ1, PPZ2 and an HAL family member. In some embodiments, the proton transport gene is selected from PMA1, PMA2 and a VMA family member.

In some embodiments, the method further comprises modifying a sodium transport gene. In some embodiments, the modified sodium transport gene encodes a polypeptide that increases the cellular efflux of sodium relative to an unmodified yeast cell. In some embodiments, the modified sodium transport gene comprises a deletion mutation or is overexpressed. In some embodiments, the modified sodium transport gene is selected from NHA1 and an ENA family member.

In some embodiments, the alcohol tolerant yeast cell is modified to comprise a deletion of PPZ1 and PPZ2 and to overexpress PMA1.

In some embodiments, the intracellular potassium of the alcohol tolerant yeast cell is maintained at a concentration of about 100 mM to about 400 mM and the intracellular pH of the alcohol tolerant yeast cell is maintained at about 5.5 to about 8.5. In some embodiments, the intracellular potassium of the alcohol tolerant yeast cell is maintained at a concentration of about 200 mM to about 300 mM. In some embodiments the intracellular pH of the alcohol tolerant yeast cell is maintained at about 7.

In some embodiments, the alcohol tolerant yeast cell is tolerant to ethanol, isopropanol and/or isobutanol.

In some embodiments, the unmodified cell is a Saccharomyces cerevisiae cell. In some embodiments, the unmodified yeast cell is of an industrial yeast cell. In some embodiments, the unmodified yeast cell is a NCYC 479 (Sake) yeast cell. In some embodiments, the unmodified yeast cell is a PE-2 (Bioethanol) yeast cell. In some embodiments, the unmodified yeast cell is an ETHANOL RED® cell.

In some embodiments, the alcohol tolerant yeast cell has been previously modified to produce ethanol, isopropanol or isobutanol.

In some embodiments, the alcohol tolerant yeast cell expresses a cellulase and/or a hemicellulase.

In some embodiments, the culturing is in culture medium that comprises fermentable feedstock. In some embodiments, the fermentable feedstock is cellulosic feedstock. In some embodiments, the fermentable feedstock is fermentable sugar. In some embodiments, the fermentable sugar is glucose. In some embodiments, the fermentable sugar is xylose. In some embodiments, the concentration of the fermentable sugar is about 50 g/L to about 400 g/L. In some embodiments, the concentration of the fermentable sugar is about 300 g/L.

In some embodiments, a plurality of the alcohol tolerant yeast cells is cultured at an OD₆₀₀ of about 15 to 50.

In some embodiments, at least 80 g/L to at least 150 g/L alcohol (e.g., ethanol) is produced. For example, in some embodiments, at least 80 g/L, at least 90 g/L or at least 100 g/L, at least 110 g/L, at least 120 g/L, at least 130 g/L, at least 140 g/L or at least 150 g/L of alcohol (e.g., ethanol) is produced. In some embodiments, at least 80 g/L to at least 150 g/L alcohol (e.g., ethanol) is produced over the course of 1 to 4 days (or at least 1 to 4 days) (e.g., 2 to 3 days), or more. For example, in some embodiments, at least 80 g/L, at least 90 g/L or at least 100 g/L, at least 110 g/L, at least 120 g/L, at least 130 g/L, at least 140 g/L or at least 150 g/L of alcohol (e.g., ethanol) is produced over the course of 1 to 4 days (or at least 1 to 4 days) (e.g., 2 to 3 days), or more.

In some embodiments, the culture medium further comprises a potassium salt, such as potassium phosphate monobasic (KH₂PO₄), potassium phosphate dibasic (K₂HPO₄) or potassium sulfate (K₂SO₄). Thus, in some embodiments, alcohol tolerant yeast cells produced by the methods as provided herein are cultured in cell culture medium that comprises a potassium salt, such as potassium phosphate monobasic (KH₂PO₄), potassium phosphate dibasic (K₂HPO₄) or potassium sulfate (K₂SO₄). In some embodiments, the potassium salt is in an amount sufficient to produce at least 80 g/L to 150 g/L (e.g., least 80 g/L, at least 90 g/L or at least 100 g/L alcohol, at least 110 g/L, at least 120 g/L, at least 130 g/L, at least 140 g/L, at least 150 g/L, or more, alcohol (e.g., ethanol). In some embodiments, culturing engineered yeast cells as provided herein in culture medium that comprises a potassium salt (e.g., KH₂PO₄, K₂HPO₄, K2SO₄) produces at least 150 g/L, or more, alcohol (e.g., at least 160 g/L or at least 170 g/L).

Other aspects of the disclosure provide a method of alcohol production, comprising culturing yeast cells (e.g., unmodified yeast cells) in culture medium that comprises fermentable feedstock and a potassium salt selected from potassium phosphate monobasic (KH₂PO₄), potassium phosphate dibasic (K₂HPO₄) and potassium sulfate (K₂SO₄), wherein the potassium salt is in an amount sufficient to produce at least 80 g/L to at least 150 g/L alcohol (e.g., ethanol) (e.g., over the course of 1 to 4 days, or at least 1 to 4 days, such as 2 to 3 days, or more). In some embodiments, the potassium salt is in an amount sufficient to produce at least 80 g/L, at least 90 g/L, at least 100 g/L, at least 110 g/L, at least 120 g/L, 130 g/L, at least 140 g/L, at least 150 g/L, or more, alcohol (e.g., ethanol) (e.g., over the course of 1 to 4 days, or at least 1 to 4 days, such as 2 to 3 days, or more). In some embodiments, the alcohol is ethanol, isopropanol or isobutanol.

In some embodiments, the potassium salt is KH₂PO₄. In some embodiments, the potassium salt is KCl and the culture medium further comprises potassium hydroxide (KOH). In some embodiments, the KOH is in an amount sufficient to maintain, in the culture medium, a pH of at least 3.5. In some embodiments, the concentration of potassium salt is about 25 mM to about 100 mM. In some embodiments, the concentration of potassium salt is about 50 mM.

In some embodiments, the fermentable feedstock is cellulosic feedstock. In some embodiments, the fermentable feedstock is fermentable sugar. In some embodiments, the fermentable sugar is glucose. In some embodiments, the fermentable sugar is xylose. In some embodiments, the concentration of the fermentable sugar is about 50 g/L to about 400 g/L. In some embodiments, the concentration of the fermentable sugar is about 300 g/L.

In some embodiments, the yeast cells are cultured at an OD₆₀₀ of about 20 to 30.

In some embodiments, the yeast cells are Saccharomyces cerevisiae cells. In some embodiments, the yeast cells are industrial yeast cells. In some embodiments, the yeast cells are NCYC 479 (Sake) yeast cells (also referred to as Kyokai 7). In some embodiments, the yeast cells are PE-2 (Bioethanol) cells. In some embodiments, the yeast cells are ETHANOL RED® cells.

In some embodiments, the yeast cells have been previously modified to produce ethanol.

In some embodiments, the yeast cells express a cellulase and/or a hemicellulase.

Still other aspects of the disclosure provide a composition comprising yeast in culture medium that comprises fermentable feedstock and a potassium salt selected from potassium phosphate monobasic (KH₂PO₄), potassium phosphate dibasic (K₂HPO₄) and potassium sulfate (K₂SO₄), wherein the potassium salt is in an amount sufficient to produce at least 80 g/L to at least 150 g/L alcohol. For example, the potassium salt may be in an amount sufficient to produce at least 80 g/L, at least 90 g/L, at least 100 g/L, at least 110 g/L, at least 120 g/L, at least 130 g/L, at least 140 g/L, at least 150 g/L, or more, alcohol (e.g., over the course of 1 to 4 days, or at least 1 to 4 days, such as 2 to 3 days, or more). In some embodiments, the yeast cells are engineered to contain a modified potassium transport gene and a proton transport gene. In some embodiments, the yeast cells are modified to express (e.g., overexpress) an enzyme that converts aldehydes into their equivalent alcohols. The enzyme may be, for example, an alcohol dehydrogenase (e.g., obtained from Saccharomyces cerevisiae or Scheffersomyces stipitis), an aldehyde dehydrogenase (e.g., obtained from Saccharomyces cerevisiae or Escherichia coli), an aldehyde reductase (e.g., obtained from Saccharomyces cerevisiae), an oxidative stress activator (e.g., obtained from Saccharomyces cerevisiae), a catalase activated by YAP1 (e.g., obtained from Saccharomyces cerevisiae), a xylose reductase (e.g., obtained from Scheffersomyces stipitis) or a methylglyoxal reductase (e.g., obtained from Escherichia coli). In some embodiments, the enzyme is an alcohol dehydrogenase (e.g., obtained from Saccharomyces cerevisiae) such as ADH1, ADH2, ADH6, ADH7 or SFA1. In some embodiments, the enzyme is an aldehyde dehydrogenase (e.g., obtained from Saccharomyces cerevisiae) such as ALD4 or ALD5. In some embodiments, the enzyme is an aldehyde reductase (e.g., obtained from Saccharomyces cerevisiae) such as GRE3 or ARI1. In some embodiments, the potassium salt is KH₂PO₄. In some embodiments, the potassium salt is KCl and the culture medium further comprises potassium hydroxide (KOH). In some embodiments, the KOH is in an amount sufficient to maintain, in the culture medium, a pH of at least 3.5. In some embodiments, the concentration of potassium salt is about 25 mM to about 100 mM. In some embodiments, the concentration of potassium salt is about 50 mM.

In some embodiments, the fermentable feedstock is cellulosic feedstock. In some embodiments, the fermentable feedstock is fermentable sugar. In some embodiments, the fermentable sugar is glucose. In some embodiments, the fermentable sugar is xylose. In some embodiments, the concentration of the fermentable sugar is about 50 g/L to about 400 g/L. In some embodiments, the concentration of the fermentable sugar is about 300 g/L.

In some embodiments, the yeast cells are cultured at an OD₆₀₀ of about 20 to 30.

In some embodiments, the yeast cells are Saccharomyces cerevisiae cells. In some embodiments, the yeast cells are industrial yeast cells. In some embodiments, the yeast cells are NCYC 479 (Sake) yeast cells. In some embodiments, the yeast cells are PE-2 (Bioethanol) cells. In some embodiments, the yeast cells are ETHANOL RED® cells.

In some embodiments, the yeast cells have been previously modified to produce ethanol.

In some embodiments, the yeast cells express a cellulase and/or a hemicellulase.

### BRIEF DESCRIPTION OF THE DRAWINGS

FIGS. 1A-1B provide graphs showing that monopotassium phosphate (K—Pᵢ, or KH₂PO₄) boosts ethanol production by enhancing tolerance. FIG. 1A shows ethanol titers (squares) and per-cell rates of ethanol production (triangles) from fermentations in unmodified medium (dashed) or medium supplemented with 50 mM KH₂PO₄/K—Pᵢ (solid). Specific productivities are calculated using the mean viable population from B during the corresponding time period. FIG. 1B shows cell densities/OD₆₀₀ (squares) and the corresponding underlying viable fractions (triangles) from the same fermentations. Error bars represent standard deviation (s.d.) from at least 3 technical replicates. FIGS. 1C and 1D provide graphs showing that elevated extracellular potassium and pH enhance ethanol tolerance and production under high glucose and high cell density conditions. FIG. 1C shows ethanol titers (squares) and per-cell rates of production (triangles) from fermentations in unmodified synthetic complete medium (YSC; dashed) or YSC supplemented with 40 mM KCl and 10 mM KOH (solid). Specific productivities are calculated from the mean viable population (thick lines from FIG. 1D) during each 24 h period. FIG. 1D shows cell densities (dry cell weight/DCW; thin squares) and the underlying viable populations (thick triangles) from the fermentations in FIG. 1C. Data are mean±SD from 3 biological replicates.

FIGS. 2A-2B provide graphs showing that K—Pᵢ enhances tolerance to alcohol shocks in high glucose. Viability after transfer from overnight growth in unmodified medium (dashed) or medium supplemented with 50 mM K—Pᵢ (solid) into identical conditions modified with the indicated concentrations of ethanol (FIG. 1A) or isopropanol (FIG. 1B). Error bars represent s.d. from at least 3 technical replicates.

FIGS. 3A-3C provide graphs showing that potassium chloride (KCl) elicits dose-dependent improvements on ethanol tolerance and production. FIG. 3A shows ethanol titers from fermentations in medium supplemented with 10-75 mM KCl. FIG. 3B shows cell densities (dashed lines) and the underlying viable fractions (solid lines) from the same fermentations. Colored areas are the respective time integrals of the viable fractions. FIG. 3C shows a correlation of the time-integrated viable fractions with final ethanol titers. Error bars represent s.d. from at least 3 technical replicates.

FIGS. 4A-4C provide graphs showing that potassium supplementation and acidity reduction recapitulate the enhancements conferred by K—Pᵢ. FIG. 4A shows ethanol titers from fermentations in medium supplemented with 50 mM K—Pᵢ (green), 50 mM KCl and periodic additions of potassium hydroxide (KOH) to approximate the pH conferred by K—Pᵢ supplementation (red), 50 mM KCl and periodic additions of KCl equimolar to the added KOH (cyan), or periodic KOH (purple) or sodium hydroxide (NaOH) (yellow) to approximate the pH conferred by K—Pᵢ supplementation. FIG. 4B shows respective cell densities (dashed) and the underlying viable components (solid). Error bars represent s.d. from at least 3 technical replicates. FIG. 4C shows a respective time course of pH. Arrows show when pH was adjusted in at least one of the three relevant fermentations (red, purple, and yellow) to approximate that conferred by K—Pᵢ supplementation; actual adjustments are indicated by jumps in pH. In conditions testing supplemental KCl, any adjustment with KOH (red) was accompanied by an addition of equimolar KCl (to cyan) to control for incremental increases in potassium. FIG. 4D shows that relative ethanol titers and statistical testing of biological triplicate fermentations conducted using YSC and the indicated supplements. Pair-wise two sample t-tests demonstrate that fermentations supplemented with 50 mM K-Pi are inseparable from those with matched potassium and pH (40 mM KCl+10 mM KOH; p=0.092), but statistically higher than those with other potassium-based salts (p≦7.58×10-3). Similarly, fermentations supplemented with 50 mM Na-Pi are indistinguishable from those with matched sodium and pH (40 mM NaCl+10 mM NaOH; p=0.217), but higher than those with NaCl alone (p≦1.96×10-4). Bivariate analysis of variance confirms that the increase conferred by potassium over sodium is significant (p=5.1×10-7), while that of phosphate vs. raised pH is insignificant (p=0.031).

FIGS. 5A-5E provide graphs showing that genetic or culture modifications modulating the potassium and proton gradients elicit corresponding effects to ethanol production or alcohol tolerance. FIG. 5A shows steady state ethanol titers from a laboratory wild-type strain (WT) and an isogenic derivative harboring a partial defect in Pma1 expression (phm4Δ). Top two bars are from unmodified medium, second two from supplementation with 50 mM K—Pᵢ, next two from supplementation with 50 mM KCl and periodic additions of KOH to approximate the pH conferred by K—Pᵢ supplementation, and final two from supplementation with 50 mM KCl and periodic additions of KCl equimolar to the added KOH. FIG. 5B provides a graph showing that genetic augmentation of the plasma membrane potassium (TRK1) and proton (PMA1) pumps increase ethanol production to levels exceeding industrial strains. Ethanol titers from a wild type laboratory strain (S288C) transformed with empty over-expression plasmid, S288C transformed with a plasmid over-expressing PMA1, S288C containing hyper-activated TRK1 (via deletions of PPZ1 and PPZ2) and transformed with empty over-expression plasmid, the TRK1 hyper-activated strain transformed with a plasmid over-expressing PMA1, and bioethanol production strains from Brazil (PE-2) and the US (Ethanol Red), all cultured in unmodified YSC lacking uracil. Data are mean±SD from 3 biological replicates. FIG. 5C shows final ethanol titers comparing unmodified medium and medium supplemented with 50 mM K—Pᵢ from a laboratory prototroph (S288C proto.), the isogenic laboratory auxotroph (S288C auxo.), NCYC 479 (Sake), and PE-2 (Bioethanol). FIG. 5D shows final ethanol titers and maximum volumetric productivity in unmodified xylose medium and xylose medium supplemented with 50 mM K—Pᵢ from strain H131-A3-AL^{CS}. FIG. 5E shows viability after transfer from overnight growth in unmodified medium (dashed) or medium supplemented with 50 mM KCl (dash-dot, solid) into the indicated conditions containing increasing concentrations of isobutanol. Error bars represent s.d. from 3 technical replicates. FIGS. 5F and 5G provide graphs showing that genetic augmentation of the plasma membrane potassium (TRK1) and proton (PMA1) pumps enhance ethanol tolerance and fermentation. FIG. 5F shows residual glucose from a wild-type laboratory strain (S288C) transformed with empty over-expression plasmid, S288C transformed with a plasmid over-expressing PMA1, S288C containing hyper-activated TRK1 (via deletions of PPZ1 and PPZ2) and transformed with empty over-expression plasmid, the TRK1 hyperactivated strain transformed with a plasmid over-expressing PMA1, and bioethanol production strains from Brazil (PE-2) and the US (Ethanol Red), all cultured in unmodified YSC lacking uracil. Corresponding ethanol titers are shown in FIG. 5B. FIG. 5G shows net fermentation viability (time integrals of the viable population) from the fermentations in FIG. 5F and FIG. 5B. Data are mean±SD from 3 biological replicates.

FIG. 6 provides a graph showing that potassium supplementation and acidity reduction enhance alcohol tolerance by strengthening the potassium and proton electrogenic gradients.

FIG. 7 provides a graph showing that K+ exerts the largest improvement in ethanol output among cations, and PO₄³⁻/Pi the largest among anions. Strain FY4/5 was fermented for 72 h in 1× yeast synthetic complete (YSC) medium containing 300 g/L glucose and the supplement indicated, all equalized for initial pH and cell density. The data are a composite of several independently conducted experiments; for comparison, maximum ethanol titers were normalized against the respective control sample containing unmodified 1×YSC.

FIGS. 8A-8B provide graphs showing that Elevated K-Pi enhances ethanol tolerance. Strain FY4/5 was fermented in 1×YSC containing 300 g/L glucose (dotted blue or black) or 1×YSC+50 mM K-Pi (solid blue or black), equalized for initial pH and cell density. FIG. 8A shows raw quantifications of the viable fraction underlying total yeast biomass (from FIG. 1B or FIG. 8B). FIG. 8B shows time-integrated areas under the curves of viable biomass (shaded) are the quantities highly correlated with final ethanol titer. The area in lighter blue, specifically, represents the net enhancing effect of supplemental K-Pi.

FIG. 9 provides a graph showing that supplemental K-Pi does not enhance ethanol fermentation by alleviating a limitation created through phosphate depletion. Extracellular phosphate concentrations are shown for FY4/5 fermented in 1×YSC (dotted black) or 1× YSC+50 mM K-Pi (solid black) containing 300 g/L glucose, both equalized for initial pH and cell density

FIGS. 10A-10B provide graphs showing that K-Pi supplementation enhances ethanol performance even when nutrients remain in abundance. Strain FY4/5 was fermented in the indicated medium conditions, all containing 300 g/L glucose and equalized for initial pH and low starting cell density (OD600≈0.2). FIG. 10A shows a time course of ethanol titer. FIG. 10B shows total yeast biomass (dotted) and the underlying viable component (solid). Plots in FIG. 10A show newly produced ethanol; starting concentrations of 3% have been subtracted from the appropriate curve.

FIGS. 11A-11B provide graphs showing that elevated K-Pi enhances ethanol fermentation via a mechanism independent of cellular phosphate homeostasis. FIG. 11A shows ethanol titers for strain BY4743 and isogenic derivatives harboring homozygous deletions of PHO4 or PHO2 after 48 h of fermentation in 1×YSC (top) or 1×YSC+50 mM K-Pi (bottom) containing 300 g/L glucose, all equalized for initial pH and cell density. FIG. 11B shows ethanol titers for BY4743 transformed with the indicated empty (WT) or overexpression plasmids after 48 h of fermentation in 1×YSC-URA containing 300 g/L glucose, all equalized for initial pH and cell density. FIGS. 11A and 11B are two independently conducted experiments; the WT baselines are not directly comparable (e.g., starting cell densities differ between the two runs).

FIGS. 12A-12C provide graphs showing that supplementation with KCl and acidity reduction with KOH can surpass the improvements conferred by elevated K-Pi. Strain FY4/5 was cultured in the indicated medium conditions, all containing 300 g/L glucose and equalized for initial pH and cell density. FIG. 12A shows a time course of ethanol titer. FIG. 12B shows total yeast biomass (dotted) and the underlying viable component (solid). FIG. 12C shows pH. Arrows in FIG. 12C indicate when KOH, or equimolar KCl as control, were added to approximate the pH conferred by elevated K-Pi.

FIGS. 13A-13B provide graphs showing that genetic augmentation of the K+ and H+ gradients elicits tolerance enhancements in the laboratory strain that match those of industrial strains. FIG. 13A shows a time course of total yeast biomass (dotted) and the underlying viable component (solid). FIG. 13B shows time integrals of the areas under the solid curves shown in FIG. 13A. Corresponding ethanol titers are shown in FIG. 5B.

FIG. 14 provides a graph showing that elevated K-Pi induces sensitivity to isobutanol in 300 g/L glucose. Strain FY4/5 was grown overnight in the indicated conditions containing 300 g/L glucose, washed to remove accumulated ethanol, and divided equally into fresh medium of the same conditions containing the indicated concentrations of isobutanol. Viability after 2.5 h was quantified by methylene blue staining and microscopy.

FIG. 15 provides a graph showing that dose-dependent permeabilization of the cell membrane to protons by ethanol is not immediately counteracted by KCl or K-Pi supplementation. Strain BY4743 (WT) was transformed with p416TEF-pHluorin and grown overnight in 1×YSC-URA containing 200 g/L glucose and any indicated supplements. Equal amounts of yeast biomass were washed and transferred into respective fresh medium containing the indicated concentrations of ethanol, incubated at room temperature for 30 min, and measured for fluorescence emission. The ratio of intensities emitted from excitation at 395 nm and 475 nm (1395/1475) is directly proportional to pH. Viability for WT at 16% ethanol, the condition expected to be most sensitized, was quantified by methylene blue staining immediately after fluorescence readings and remains at a maximum (e.g., fluorescence readings were not impacted by non-viable cells).

FIGS. 16A-16C provide graphs showing that supplemental KCl and K-Pi enhance ethanol performance under increasing glucose load. Strain FY4/5 was fermented for 72 h in the indicated medium conditions, all equalized for initial pH and cell density. FIG. 16A shows maximum volumetric ethanol titers. FIG. 16B shows maximum volumetric productivities. FIG. 16C shows percentages of theoretical yield ([g ethanol/g glucose/0.51×100]).

FIG. 17 provides a graph showing the viability of yeast cells over time when cultured in culture medium comprising 13% ethanol or 13% ethanol and 50 mM K-Pi.

FIGS. 18A-18E provide graphs showing that elevated potassium and pH are sufficient to enhance tolerance independently of strain genetics, sugar substrate, and alcohol species. FIG. 18A shows ethanol titers from glucose fermentation (top) of one laboratory (S288C) and three industrial (PE-2, Ethanol Red, Kyokai 7) yeast strains, or from xylose fermentation (bottom) of an engineered xylose strain, in unmodified YSC or YSC supplemented with 40 mM KCl and 10 mM KOH (designated herein, in some instances, as “40/10 mM KCL/KOH”). FIG. 18B shows titers from S288C cultured in 20% yeast extract-peptone medium (YP) or supplemented with potassium at pH 6 and 3.7. FIG. 18C shows population fractions of S288C after transfer from overnight growth in unmodified YSC (dashed), or that supplemented with 48 mM KCl and 2 mM KOH (solid), into media containing the indicated concentrations of ethanol. FIGS. 18D and 18E are similar to FIG. 18C, but with step increases of isopropanol or isobutanol, respectively. All data are mean±SD from 3 biological replicates.

FIG. 19 provides a graph showing that elevated potassium and pH are sufficient to induce complete consumption of fermentation sugar independently of strain genetics and sugar substrate. Residual sugar from glucose fermentation (top) of one laboratory (S288C) and three industrial (PE-2, Ethanol Red, Kyokai 7) yeast strains, or from xylose fermentation (bottom) of an engineered xylose strain, grown in unmodified YSC or YSC supplemented with 40 mM KCl and 10 mM KOH. Corresponding ethanol titers are shown in FIG. 18A. Data are mean±SD from 3 biological replicates.

FIGS. 20A and 20B provide graphs showing that elevated potassium is sufficient to enhance fermentation in chemically undefined medium containing yeast extract and peptone (YP). FIG. 20A shows ethanol titers from S288C cultured in undiluted YP, YP diluted to 30%, or YP diluted to 3%, all containing 300 g/L glucose and supplemented with either 50 mM potassium (as KCl) or calcium (as CaCl₂). FIG. 20B shows residual glucose from the fermentations in FIG. 20A. Data are mean±SD from three biological replicates.

FIGS. 21A and 21B provide graphs showing that genetic impairment of potassium import or proton export decreases ethanol performance. FIG. 21A shows ethanol titers from an auxotrophic wild type laboratory strain (S288C-based BY4743), an isogenic derivative harboring a homozygous deletion of the potassium pump (trk1Δ/trk1Δ), and an isogenic derivative with a heterozygous deletion of the proton pump (PMA1/pma1Δ), all cultured in unmodified YSC (top) or YSC supplemented with 40 mM KCl and 10 mM KOH (bottom). FIG. 21B show residual glucose from the fermentations in FIG. 21A. Data are mean±SD from 3 biological replicates.

FIG. 22A provides a graph showing a comparison of ethanol production in YSC medium supplemented with 300 g/L glucose and 40/10 mM KCL/KOH in bioreactors with aeration and under anaerobic conditions. FIGS. 22A and 22B provide graphs showing elevated potassium and pH enhance ethanol production in an anaerobic bioreactor environment. FIG. 22B shows a time course of ethanol production (black solid), glucose consumption (black dashed), and pH (blue). Manual additions of 2 mM KOH are indicated by blue arrows. FIG. 22C shows corresponding time course of cell density (dashed) and the underlying viable cell population (solid).

FIGS. 23A and 23B provide graphs showing that elevated K⁺ and pH can overcome cellular toxicity in acid hydrolysates of cellulosic biomass.

FIG. 24 provides a graph showing that KCl/KOH confer cellular tolerance of heat.

### DESCRIPTION OF THE INVENTION

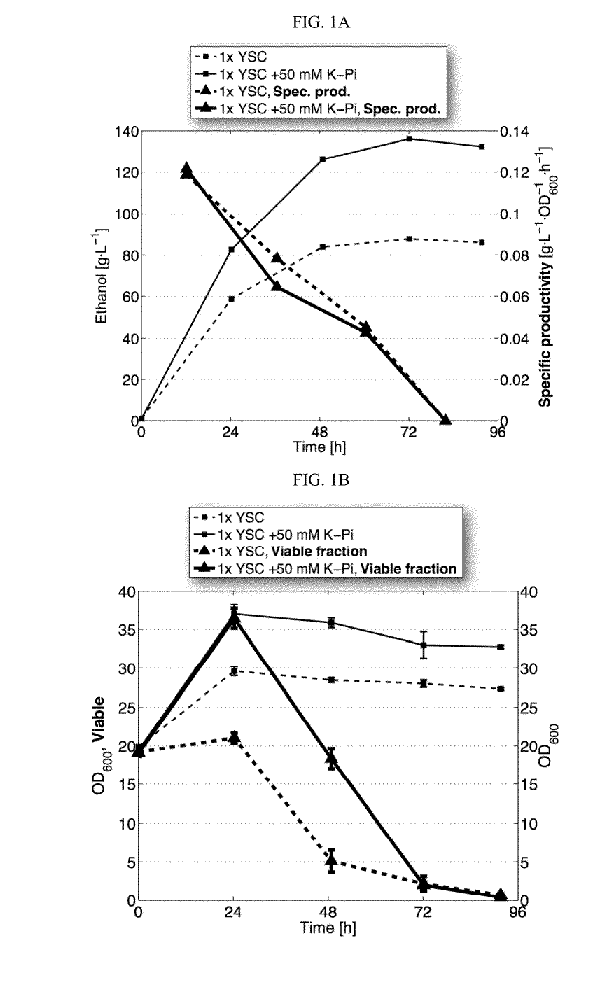

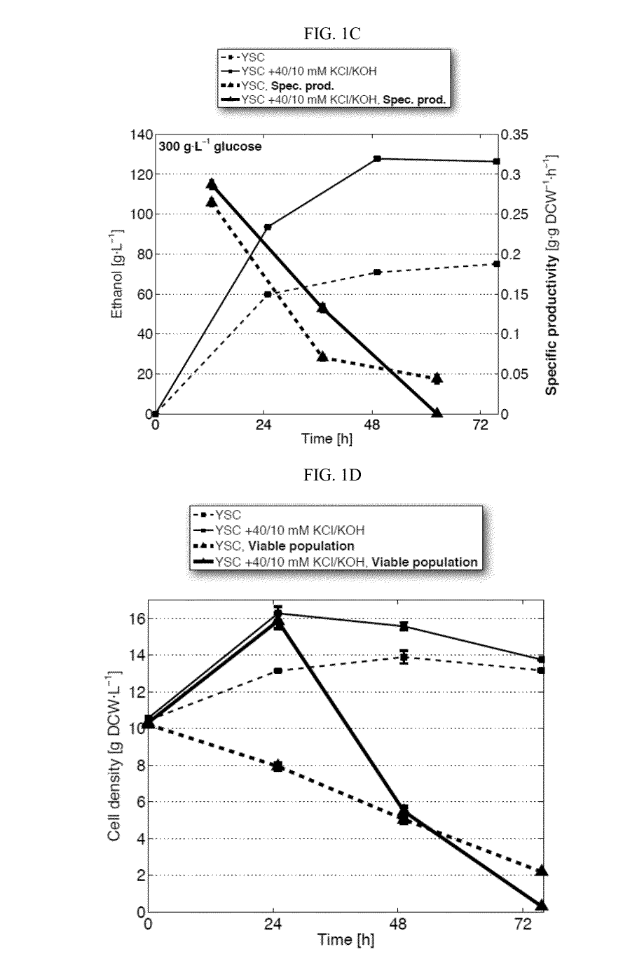

Alcohol fermentation such as, for example, ethanol fermentation, is the process by which sugars/monosaccharides (e.g., glucose) are converted into alcohol and carbon dioxide by organisms such yeast. Thus, alcohol tolerance in yeast is an important factor in regulating the level of alcohol than can be produced during the fermentation process. The present disclosure shows that membrane gradients can have a fundamental and strain-independent role in determining alcohol (e.g., ethanol) tolerance. Thus, provided herein, in some embodiments, are genetic modifications aimed at strengthening the ion pump activities responsible for establishing the K⁺ and H⁺ gradients, which can elicit corresponding improvements to ethanol production. This disclosure presents a toxicity model where alcohols attack viability not at threshold concentrations that solubilize lipid bilayers, but at lower concentrations that increase permeability of the plasma membrane and disrupt a cell's ionic membrane gradients. In yeast, the coupled ATP-dependent import of K⁺ and export of H⁺ generate a major component of the electrical membrane potential, which is used to power a variety of the cell's exchange processes with the environment. Without being bound by theory, a possible mode of cell death during fermentation arises from the breakdown of transport of essential nutrients and waste products, and may occur long before ethanol accumulates to levels that chemically destroy the membrane bilayer. Several lines of evidence provided herein support this hypothesis. First, fermentations conducted with elevated potassium phosphate monobasic (K—Pᵢ) demonstrated that yeast are generally capable of withstanding ethanol concentrations above 100 g/L; thus, the sub-100 g/L titers reached in fermentations performed in unmodified medium represent a biological, rather than chemical, limit (FIGS. 1A, 1B). Second, shocks of increasing ethanol concentration decreased intracellular pH in a dose-dependent fashion, demonstrating that ethanol permeabilizes the plasma membrane to protons and potentially other ions (FIG. 15). The outward-facing H⁺ gradient, therefore, is likely disrupted with increasing strength as ethanol accumulates during the course of fermentation. A rise in cytosolic acidity, however, is unlikely to be the direct cause of cell death as K—Pᵢ and KCl supplementation have both been shown to improve viability yet are not capable of counteracting the ethanol-mediated pH drop (FIG. 15). Given its amphipathicity, ethanol progressively increases the leakage of ions, requiring the cell to expend escalating amounts of energy to reestablish the steep separation of charges.

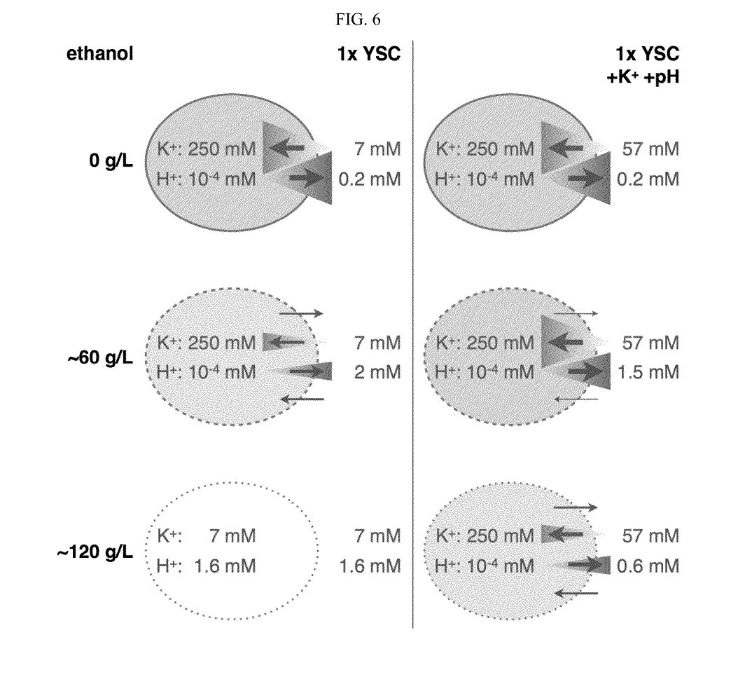

Conditions that bolster the cell's efforts to maintain the high concentrations of intracellular K⁺ (e.g., 200-300 mM) and low intracellular H⁺ (e.g., ˜pH 7) thus enhance tolerance by raising the threshold to which alcohols will collapse these drivers of homeostasis (FIG. 6). The present disclosure shows that physical reinforcements in the form of ionic adjustments to the medium (for example, supplemental K—Pᵢ, or supplemental KCl and KOH) generate the greatest overall improvements. Not only do higher concentrations of extracellular K⁺ assist import (e.g., pumping against a ˜4 fold higher gradient vs. ˜36 fold), and lower concentrations of extracellular H⁺ assist export, the corresponding rates of ion leakage from the cell are lowered due to the reduced differentials.

Thus, provided herein are alcohol tolerant yeast cells engineered to maintain, in the presence of alcohol, a high concentration of intracellular potassium and a low intracellular pH. An “engineered” yeast cell refers to a yeast cell that is modified to contain a recombinant or synthetic nucleic acid. An engineered yeast cell is not a naturally-occurring cell. As used herein, an “alcohol tolerant yeast cell” refers to an engineered yeast cell with increased viability relative to an unmodified cell (e.g., wild-type cell) when cultured in the presence of alcohol. It should be understood that, in some instances, the alcohol tolerance (e.g., viability) of a yeast cell may depend on a combination of factors such as, for example, the alcohol concentration and the fermentable sugar concentration in which the yeast cell is cultured. For example, an engineered yeast cell that remains viable for a period of time that is at least (or about) 3-fold greater relative to an unmodified yeast cell when cultured for at least 3 hours in culture medium with an alcohol concentration of about 13% and a glucose concentration of about 300 g/L is considered herein to be an alcohol tolerant yeast cell. As another example, an engineered yeast cell that remains viable for a period of time that is at least (or about) 5-fold greater relative to an unmodified yeast cell when cultured under the same conditions for at least 6 hours in culture medium with an alcohol concentration of about 13% and a glucose concentration of about 300 g/L is considered herein to be an alcohol tolerant yeast cell.

In some embodiments, an alcohol tolerant yeast cell is viable for a defined period of time in culture medium with an alcohol concentration of about 100 g/L to about 500 g/L and a fermentable sugar concentration of about 50 g/L to about 400 g/L. In some embodiments, an alcohol tolerant yeast cell is viable for a defined period of time in culture medium with an alcohol concentration of less than 100 g/L (e.g., 70 g/L, 80 g/L or 90 g/L).

In some embodiments, the defined period of time in which an alcohol tolerant yeast cell is viable in the presence of alcohol is at least 3 hours, at least 3.5 hours, at least 4 hours, at least 4.5 hours, at least 5 hours, at least 5.5 hours, at least 6 hours, at least 6.5 hours, at least 7 hours, or more.

In some embodiments, the alcohol concentration of the cell culture medium is at least 70 g/L, 80 g/L, 90 g/L, 100 g/L, 110 g/L, 120 g/L, 130 g/L (or 13%), 140 g/L (or 14%), 150 g/L (or 15%), 160 g/L (or 16%), 170 g/L (or 17%), 180 g/L (18%), 190 g/L (19%), 200 g/L (20%), or more (e.g., of culture medium). In some embodiments, the alcohol concentration of the cell culture medium is about 100 g/L (or 10%) to about 200 g/L (or 20%) (e.g., of culture medium). For example, in some embodiments, the alcohol concentration of the cell culture medium is about 100 g/L, about 110 g/L, about 120 g/L, about 130 g/L, about 140 g/L, about 150 g/L, about 160 g/L, about 170 g/L, about 180 g/L, about 190 g/L, or about 200 g/L. In some embodiments, the alcohol concentration is more than 200 g/L.

In some embodiments, alcohol is produced at a concentration of at least 70 g/L, 80 g/L, 90 g/L, 100 g/L, 110 g/L, 120 g/L, 130 g/L (or 13%), 140 g/L (or 14%), 150 g/L (or 15%), 160 g/L (or 16%), 170 g/L (or 17%), 180 g/L (18%), 190 g/L (19%), 200 g/L (20%), or more (e.g., of culture medium) over the course of 1 to 4 days (or at least 1 to 4 days), or more (e.g., 1 day, 2 days, 3 days, 4 days, or more), or 1 to 2 days, 1 to 3 days, 2 to 3 days, 2 to 4 days, or 3 to 4 days. In some embodiments, the alcohol concentration of the cell culture medium is about 100 g/L (or 10%) to about 200 g/L (or 20%) (e.g., of culture medium) over the course of 1 to 4 days (or at least 1 to 4 days) (e.g., 1 day, 2 days, 3 days, 4 days, or more). In some embodiments, the alcohol concentration of the cell culture medium is about 100 g/L (or 10%) to about 200 g/L (or 20%) (e.g., of culture medium) over the course of 1 to 2 days, 1 to 3 days, 2 to 3 days, 2 to 4 days, or 3 to 4 days. For example, in some embodiments, the alcohol concentration of the cell culture medium is at least or about 100 g/L, at least or about 110 g/L, at least or about 120 g/L, at least or about 130 g/L, at least or about 140 g/L, at least or about 150 g/L, at least or about 160 g/L, at least or about 170 g/L, at least or about 180 g/L, at least or about 190 g/L, or at least or about 200 g/L. In some embodiments, the alcohol concentration is more than 200 g/L over the course of at 1 to 4 days (or at least 1 to 4 days), or more (e.g., 1 day, 2 days, 3 days, 4 days, or more). In some embodiments, the alcohol concentration is more than 200 g/L over the course of 1 to 2 days, 1 to 3 days, 2 to 3 days, 2 to 4 days, or 3 to 4 days.

In some embodiments, the fermentable sugar concentration of the cell culture medium is about 50 g/L to about 400 g/L (e.g., of culture medium). For example, in some embodiments, the fermentable sugar concentration of the cell culture medium is about 50 g/L, about 100 g/L, about 150 g/L, about 200 g/L, about 250 g/L, about 300 g/L, about 350 g/L or about 400 g/L. In some embodiments, the fermentable sugar concentration is more than 400 g/L.

It should also be understood that yeast cells described herein, in some embodiments, may be tolerant to alcohol when cultured in culture medium that is adjusted for potassium (K⁺) and pH, as described elsewhere herein. Thus, in some embodiments, unmodified yeast cells may be tolerant to alcohol when cultured in culture medium adjusted for K⁺ and pH.

In some embodiments, modified yeast cells are cultured in culture medium that is adjusted for potassium (K⁺) and pH, as described elsewhere herein. For example, yeast cells engineered to comprise a modified potassium transport gene encoding a polypeptide that increases cellular influx of potassium relative to an unmodified yeast cell and a modified proton transport gene encoding a polypeptide that increases the cellular efflux of protons relative to an unmodified yeast cell may be cultured in culture medium that is adjusted for potassium (K⁺) and pH. In some embodiments, yeast cells are also engineered to express an enzyme that converts aldehydes into their equivalent alcohols.

Any yeast capable of fermentation may be used (e.g., modified and/or cultures) as provided herein. Examples of yeast strains for use in accordance with the present disclosure include, without limitation, the following: Saccharomyces spp., Schizosaccharomyces spp., Scheffersomyces spp. Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. In some embodiments, the yeast strain is a Saccharomyces cerevisiae (S. cerevisiae) strain. In some embodiments, the yeast strain is an industrial yeast strain (S. cerevisiae strain) used in bioethanol production. An “industrial” yeast strain, as used here, refers to a yeast strain used in the commercial production of alcohol (e.g., ethanol). In some embodiments, an industrial yeast strain is a polyploid strain that has been selected over time for alcohol (e.g., ethanol) productivity and tolerance to alcohol, temperature and/or sugar. For example, in some embodiments, the yeast strain is a sake yeast strain (e.g., strains of Saccharomyces cerevisiae such as NCYC 479/Kyokai no. 7), PE-2 (Argueso J L et al. Genome Res. 19(12), 2258-70 (2009), incorporated by reference herein) or ETHANOL RED® (LeSaffre Corporations, Fermentis). Other examples of industrial yeast strains include NCYC 73, NCYC 177, NCYC 431, NCYC 478, NCYC 975 and NCYC 1236.

An engineered yeast cell with a “high concentration of intracellular potassium” herein refers to an engineered yeast cell with an intracellular potassium concentration of at least 100 mM. “Intracellular potassium” refers to the concentration of potassium ions (K⁺) inside a cell. In some embodiments, the intracellular potassium concentration of an engineered yeast cell is at least 200 mM. In some embodiments, the intracellular potassium concentration of an engineered yeast cell is about 100 mM to about 500 mM. For example, in some embodiments, intracellular potassium concentration of an engineered yeast cell is about 100 mM, about 150 mM, about 200 mM, about 250 mM, about 300 mM, about 350 mM, about 400 mM, about 450 mM, about 500 mM, or more. In some embodiments, the intracellular potassium concentration of an engineered yeast cell is about 200 mM to about 300 mM.

An engineered yeast cell with a “low intracellular pH” herein refers to an engineered yeast cell with in intracellular pH of about 5.5 to about 8.5. “Intracellular pH” refers to the measure of acidity or basicity of the aqueous environment inside a cell, which reflect the concentration of protons (H⁺), or hydrogen ions, inside the cell. In some embodiments, the intracellular pH of an engineered yeast cell is about 7.

The alcohol tolerant yeast cells provided herein may be engineered to comprise a modified potassium transport gene encoding a polypeptide (e.g., protein) that increases cellular influx of potassium relative to an unmodified yeast cell and a modified proton transport gene encoding a polypeptide that increases the cellular efflux of protons relative to an unmodified yeast cell. “Cellular influx” of potassium refers to a process by which potassium ions are transported across a cell membrane into the intracellular compartments of a cell. “Cellular efflux” of protons refers to a process by which protons are transported across a cell membrane out of a cell into extracellular space.

An “unmodified yeast cell,” as used herein, refers to a yeast cell that is not engineered such as, for example, a wild-type yeast cell.

A “potassium transport gene,” as used herein, refers to a gene encoding a polypeptide that functions in the process of moving potassium ions (K⁺) across a cell membrane. Potassium transport genes includes those genes encoding polypeptides that directly regulate potassium ion transport across a cell membrane as well as those genes encoding polypeptides that indirectly regulate potassium ion transport. For example, the TRK1 encodes an ATP-driven K⁺ transporter membrane protein required for high-affinity potassium transport in yeast; thus, TRK1 is considered herein to be a potassium transport gene encoding a polypeptide that directly regulates potassium ion transport. Comparatively, deletion of phosphatases PPZ1 and PPZ2 have been reported to result in hyperactivation of TRK1; thus, PPZ1 and PPZ2 are considered herein to be potassium transport genes encoding polypeptides that indirectly regulate potassium ion transport. Other examples of potassium transport genes include, without limitation, TRK2, which encodes an ATP-driven K⁺ transporter membrane protein, and HAL family members (e.g., HAL1, HAL3, HAL4, HAL5), which encode proteins that regulate TRK-encoded K⁺ transporters.

A “proton transport gene,” as used herein, refers to a gene encoding a polypeptide that functions in the process of moving protons (H⁺) across a cell membrane. Proton transport genes include those genes encoding polypeptides that directly regulate proton transport across a cell membrane as well as those genes encoding polypeptides that indirectly regulate proton transport. For example, PMA1 encodes an H⁺ transporter membrane protein required for proton transport in yeast; thus, PMA1 is considered herein to be a proton transport gene encoding a polypeptide that directly regulates proton transport. Comparatively, RAP1 and GCR1 are transcriptional activators of PMA1; PKT2 and YCK1/YCK2 phosphorylate PMA1; HSP30 inhibits PMA1 under heat shock conditions; and STD1 can form a complex with PMA1; thus, RAP1, GCR1, PKT2, YCK1, YCK2, HSP30 and STD1 are considered herein to be proton transport genes encoding polypeptides that indirectly regulate proton transport. Other examples of potassium transport genes include, without limitation, PMA2, which encodes an H⁺ transporter membrane protein, and VMA family members (e.g., VMA1, VMA2, VMA3, VMA7, VMA8, VMA9, VMA10), which encode proteins that regulate vacuolar H⁺ transporter proteins.

The alcohol tolerant yeast cells provided herein may, in some embodiments, be engineered to comprise a modified sodium transport gene. A “sodium transport gene,” as used herein, refers to a gene encoding a polypeptide that functions in the process of moving sodium ions (Na⁺) across a cell membrane. Sodium transport genes include those genes encoding polypeptides that directly regulate sodium transport across a cell membrane as well as those genes encoding polypeptides that indirectly regulate sodium transport. For example, ENA family members encode a Na⁺ transporter membrane protein required for sodium transport in yeast; thus, ENA family members (e.g., ENA1, ENA2, ENA3, ENA4, ENA5, ENA6) are considered herein to be sodium transport genes encoding polypeptides that directly regulates sodium transport. In some embodiments, an alcohol tolerant yeast cell is engineered to comprise a modified sodium transporter gene encoding a polypeptide that increases the cellular efflux of sodium relative to an unmodified cell. “Cellular efflux” of sodium refers to a process by which sodium ions are transported across a cell membrane out of a cell into extracellular space.

In some embodiments, an alcohol tolerant yeast cell is engineered to comprise modified NHA1, which encodes a membrane protein that catalyzes the exchange of H⁺ for Na⁺ in a manner that is dependent on pH.

In some embodiments, an alcohol tolerant yeast cell is engineered to express (e.g., overexpress) an enzyme that converts aldehydes into their equivalent alcohols (e.g., an alcohol dehydrogenase that converts furfural to furfuryl alcohol). Such enzymes confer to yeast cells tolerance in cellulosic hydrolysates, for example. Surprisingly, the results from experiments described herein demonstrate that elevated K⁺ and pH can overcome the toxicity associated with acid hydrolysates of cellulosic biomass. As shown in Example 11, elevated K⁺ and pH in cell culture medium supplemented with known inhibitors (e.g., acetic acid, furfural, and hydroxymethylfurfural (HMF)) enhanced alcohol production. Thus, the present disclosure contemplates converting inhibitors, such as furfural and HMF, into their equivalent alcohols and combining this conversion process with K⁺/pH supplementation or genetic modification of K⁺/pH pumps to enhance cellulosic ethanol production in yeast cells.

Enzymes that convert aldehydes into their equivalent alcohols may be obtained from yeast or bacteria, for example. In some embodiments, the enzyme is obtained from Saccharomyces cerevisiae (e.g., ADH1, ADH2, ADH6, ADH7, SFA1, ALD4, ALD5, GRE3, ARI1, YAP1, CTA1 and/or CTT1) or Scheffersomyces stipitis (e.g., ADH4, ADH6 and/or XYL1). In some embodiments, the enzyme that converts aldehydes into their equivalent alcohols is obtained from Escherichia coli (e.g., YqhD and/or DkgA). In some embodiments, the enzyme that converts aldehydes into their equivalent alcohols is obtained from Cupriavidus basilensis, Burkholderia phytofirmans, Burkholderia phymatum, Bradyrhizobium japonicum and/or Methylobacterium radiotolerans (e.g., hmfABCDE and/or hmfFGH).

Examples of enzymes that convert aldehydes into their equivalent alcohols include, without limitation, alcohol dehydrogenases (e.g., ADH1, ADH2, ADH6, ADH7 and SFA1 from Saccharomyces cerevisiae, and ADH4 and ADH6 from Scheffersomyces stipitis), aldehyde dehydrogenases (e.g., ADL4 and ADL5 from Saccharomyces cerevisiae, and YqhD from Escherichia coli), aldehyde reductases (e.g., GRE3 and ARI1 from Saccharomyces cerevisiae), oxidative stress activators (e.g., YAP1 from Saccharomyces cerevisiae), catalases activated by Yap1 (e.g., CTA1 and CTT1 from Saccharomyces cerevisiae), xylose reductases (e.g., XYL1 from Scheffersomyces stipitis), methylglyoxal reductase (e.g., DkgA from Escherichia coli), and enzymes from the furfural and HMF metabolism clusters (e.g., hmfABCDE, hmfFGH).

A “modified” gene, as used herein, refers to a gene that is mutated, overexpressed or misexpressed. In some embodiments, the mutation is a deletion mutation, or a deletion. A “deletion mutation” refers to a region of a chromosome that is missing (i.e., loss of genetic material), which affects the function of a gene, or gene product (e.g., polypeptide encoded by the gene). Any number of nucleotides can be deleted. In some embodiments, a deletion mutation may render a gene, or gene product, non-functional. The symbol “A” denotes a deletion mutation. For example, engineered ppz1Δ/ppz2Δ yeast have a deletion mutation in PPZ1 and PPZ2. Methods of introducing genetic mutations in yeast are well-known, any of which may be used in accordance with the present disclosure (Sherman, F. in Encyclopedia of Molecular Biology and Molecular Medicine (Meyers, R. A.) 6, 302-325 (Wiley-Blackwell, 1998); Orr-Weaver, T. L., et al. Proc Natl Acad Sci USA 78, 6354-6358 (1981); Sikorski, R. S. & Hieter, P. Genetics 122, 19-27 (1989); and Wach, A., et al. Yeast 10, 1793-1808 (1994), each of which is incorporated by reference herein). A modified gene, or gene product, is herein considered to be “overexpressed” if the expression levels of the gene, or gene product, are increased relative to the expression levels of an unmodified (e.g., wild-type) gene, or gene product. A modified gene, or gene product, is herein considered to be “misexpressed” if the gene, or gene product, is expressed at a cellular location where or at a developmental time when it is not normally expressed. Methods of overexpression and misexpression in yeast are well-known, any of which may be used in accordance with the present disclosure (Mumberg, D., et al. Gene 156, 119-122 (1995); Mumberg, D., et al. Nucleic Acids Res 22, 5767-5768 (1994); and Avalos, J. L., et al. Nat Biotechnol 31, 335-341 (2013), each of which is incorporated by reference herein).

Ethanol resistance is increased substantially and concomitantly with ethanol production under the high sugar (e.g., 300 g/L) and high cell density (e.g., OD600˜20-30) conditions that are typical of large-scale industrial fermentation. As used herein, “industrial fermentation” refers to the use of fermentation by yeast to produce useful products such as biofuel (e.g., ethanol, or bioethanol). A fermentation process (e.g., conversion of sugar to alcohol) is herein considered to be “large-scale” if the process includes culturing fermenting yeast cells (e.g., engineered yeast cells) in a volume of at least 5 liters (L) (e.g., of culture medium). In some embodiments, a large-scale industrial fermentation process may include culturing fermenting yeast cells in a volume of at least 10 L, at least 15 L, at least 20 L, at least 25 L, at least 50 L, at least 100 L, at least 500 L, at least 1,000 L, at least 5,000 L or at least 10,000 L. In some embodiments, a large-scale industrial fermentation process may include culturing fermenting yeast cells in a volume of at least 100,000 L, at least 500,000 L, or at least 1,000,000 L. The yeast cells may be cultured in, for example, shake flask cultures or bioreactors.

Industrial fermentation processes may also include culturing yeast in the presence of a high concentration of fermentable feedstock or fermentable sugar. “Fermentable feedstock” herein refers to feedstock that can be converted (e.g., by yeast) to sugar and then to alcohol. Non-limiting examples of a fermentable feedstock include lignocellulosic biomass (e.g., (corn stover, sugarcane bagasse, straw), composed of carbohydrate polymers (e.g., cellulose, hemicellulose) and an aromatic polymer (e.g., lignin) A “fermentable sugar” herein refers to a sugar that can be converted (e.g., by yeast) to alcohol. Examples of fermentable sugars for use in accordance with the present disclosure include, without limitation, allose, altrose, glucose, mannose, gulose, idose, galactose, talose, psicose, fructose, sorbose, tagatose, arabinose, lyxose, ribose, xylose, ribulose and xylulose. Sources of fermentable sugars include, without limitation, feedstock such as corn, wheat, sorghum, potato, sugarbeet, sugarcane, potato-processing residues, sugarbeet, cane molasses and apple pomace. Fermentable sugars can be produced directly or derived from polysaccharides such as cellulose and starch. In some embodiment, the fermentable sugar is from (e.g., derived from) a lignocellulosic substance. Thus, in some embodiments, the fermentable sugar is a hexose such as glucose. In some embodiments, the fermentable sugar is from xylan hemicellulose. Xylose can be recovered by acid or enzymatic hydrolysis. Thus, in some embodiments, the fermentable sugar is a pentose such as xylose. Enzymatic hydrolysis using mixtures of enzymes, such as cellulase and hemicellulases, may be used herein to avoid the destruction of sugars associated with acid treatments (hydrolysis) of lignocellulosic material. These enzymes, when combined with effective pretreatment of lignocellulosics, provide high yields of glucose, xylose, and other fermentable sugars with minimal sugar losses. In some embodiments, the engineered yeasts strains provided herein also express a cellulase and/or a hemicellulase. Examples of cellulases that may be expressed by the yeast cells and/or engineered yeast cells are provided in Table 1, and examples of hemicellulases that may be expressed by the yeast cells and/or engineered yeast cells are provided in Table 2. Other examples of cellulases and hemicellulases are described in Zyl, W. H., et al. Adv. Biochem. Eng. Biotechnol. 108, 205-235 (2007), incorporated by reference herein. In some embodiments, the yeast cells and/or engineered yeast cells may express a combination of cellulase(s) and hemicellulase(s) provided in Tables 1 and 2.

TABLE 1
Cellulase components expressed in S. cerevisiae.

| | | | Substrate(s) activity was | |
|---|---|---|---|---|
| | | | detected against (values | Specific |
| Organism & | Titer | % cell | indicate activity measured | activity |
| gene/enzyme | (mg/L) | protein | per L culture broth) | (U/mg) |
| CBHI | | | | |
| Trichoderma reesel CBHI | 2 | 1.5 | MUC, AC | NR |
| | 5 | 0.123 | MUL, BMCC | 0.26 |
| | | | | (on BMCC) |
| | 0.22 | 0.006 | 0.06 U/L (PASC), | 0.22 |
| | | | 0.06 U/L (BMCC) | (on PASC) |
| Aspergillus niger CBHB | NR | NR | 0.035 U/L (AC), | NR |
| | | | 0.03 U/L (BMCC) | |
| Pitanerochaete | NR | NR | 12 U/L, ~3.3 U/g | NR |
| chryrosporium CBHI-4 | | | DCW (BBG), 10 U/g DCW | |
| | NR | NR | (PNPC) 22 U/g DCW (AC) | NR |
| | NR | NR | 18 U/g DCW (PNPC) | NR |
| | NR | NR | 0.035 U/L (AC), | NR |
| | | | 0.03 U/L (BMCC) | |
| Poticillium | NR | NR | MUL | NR |
| janthinellum CBHI | | | | |
| Thera occurs | 0.1 | 0.002 | Avicel, AC, PNPC, PNPL | 0.03, 0.04, |
| aurare tacus CBHI | | | | 0.11, 0.29 |
| | | | | (same order |
| | | | | as activity) |
| Aspergillus | 7 | 0.173 | Avicel, MUL | 0.007 |
| aculeatus CBHI | | | | (Avicel) |
| Cellulomonas fimi cex | 2.5 | 0.03 | 8 U/L, | 3 |
| | | | ~1.0 U/g DCW (PNPC) | (on PNPC) |
| Cellulomonas fimi Exg | 12.5 | NR | 45 U/L (PNPC) | 3.6 (PNPC) |
| (cex) | | | | |
| CBHII | | | | |
| Trichoderma reesel | 100 | 2.6 | BBG, AC | NR |
| CBHII | | | | |
| | 10 | 0.33 | 24 U/L, 3 U/g DCW (AC) | 0.7 (on AC) |
| | NR | NR | 0.15 U/g DCW (AC) | NR |
| | NR | NR | 0.34 U/L (AC), | NR |
| | | | 0.09 U/L (BMCC) | |
| Agaricus bisporus CELS | NR | NR | 0.06 U/g DCW (AC), | NR |
| | | | 0.033 U/g DCW (CC), | |
| | | | 0.008 U/g DCW (BBG) | |
| EG | | | | |
| Trichoderma reesel EGI | NR | 0.5 | CMC | 15 |
| | | | | (on CMC) |
| | 10 | 0.09 | MUC | NR |
| | 0.66 | 0.25 | BBG, lichenan, CMC, | NR |
| | | | HEC, MUL, MUC | |
| | 5 | 0.12 | 72 U/g DCW (HEC) | 60 (on HEC) |
| Trichoderma racsei EGII | NR | NR | 3.64 U/g DCW (AC) | NR |
| Trichoderma racsei EGIII | NR | NR | BBG, lichenan, | NR |
| | | | CMC, HEC | |
| Trichoderma racsei EGV | NR | NR | BBG, HEC | NR |
| Trichoderma racsei EGIV | NR | NR | BBG, AC, CMC | NR |
| Aspergillus niger engl | 2.8 | 0.07 | 574 U/L (CMC) | 204 |
| | | | | (on CMC) |
| Aspergillus aculeatus | NR | NR | 0.5 U/L | NR |
| CMCase | | | ~0.06 U/g DCW (CMC) | |
| Aspergillus aculeatus | NR | NR | 60 U/L (CMC) | NR |
| F1-CMCase | | | | |
| | NR | NR | CMC, IOSC | H |
| | | | | (on IOSC) |
| Cellulamonas fimi Eng | 13 | NR | 293 U/L (low viscosity | NR |
| (cenA) | | | CMC) | |
| Cellulamonas fimi | NR | NR | 1600 U/L (CMC) | NR |
| CMCase | | | | |
| Thermoascus | 1.5 | 0.04 | 197 U/mg total protein, | 336 |
| auransfacus egl | | | ~535 U/L (CMC) | (on CMC) |
| Cryptococcus flavus | NR | NR | 12 500 U/L | NR |
| CMCI | | | ~1,390 U/g DCW (CMC) | |
| Clostridium | NR | NR | 280 U/g 24 U/g DCW | NR |
| thermocellium colA | | | (CMC) | |
| Clostridium | NR | NR | 2000 U/g total protein | NR |
| thermocellum EG (colA) | | | (CMC) | |
| Butyrivibrio | NR | NR | 22 U/g DCW (AC) | NR |
| fibrisolvens ENDI | | | | |
| | NR | NR | 4.3 U/g DCW (BBG) | NR |
| | NR | NR | 1100 U/L | NR |
| | | | ~300 U/g DCW (BBG) | |
| | NR | NR | 3,460 U/L CMC) | NR |
| | NR | NR | BBG | NR |
| Scopulariopsis | NR | NR | 109 U/L | NR |
| breviaralis EGI | | | ~12.1 U/L DCW (CMC) | |
| Bacillus circulans Endol | NR | NR | 300 U/L | NR |
| Exo bifunctional enzyme | | | ~33 U/g DCW (CMC) | |
| Trichoderma | NR | NR | azo-BBG | NR |
| longibruchiatum egl1 | | | | |
| Bascillus substilius endo- | NR | NR | 33600000 U/L (BBG) | NR |
| beta-1,3-1,4-glucanase | | | | |
| | NR | NR | 2.3 U/g total protein (BBG) | NR |
| Bacillus substilis BEGI | NR | NR | BBG | NR |
| Bacillus substilis EG | NR | NR | 1650 U/L (CMC) | NR |
| Thermoanaerobacter | NR | NR | 26 U/L (CMC) | NR |
| cellulolyticus | | | | |
| Endoglucanase | | | | |
| Cellulomortas | NR | NR | 167 U/L (CMC) | NR |
| biazotea EG | | | | |
| Acidothermus cellulolyticus | NR | NR | 1700 000 U/g | NR |
| E1 beta-1,4- | | | total protein (MUC) | |
| endo-glucanase | | | | |
| Trichoderma | NR | NR | azo-BBG | NR |
| longibrachiatum EG | | | | |
| Barley 1,3-1,4-beta- | NR | NR | BBG | NR |
| glucanase | | | | |
| BGL | | | | |
| Kluyveromyces | NR | 15 | PNPG, C2 | 64.4 |
| fragllis BGL | | | | (on PNPG) |
| Aspergillus acculeatus | NR | NR | BGL1 = 21.3 U/g DCW | NR |
| BGLI | | | (PNPG) | |
| | 1 | 0.02 | IOSC | 25 |
| | | | | (on IOSC) |
| Sacoharomycopsis | 10 | 0.25 | PNPG, C2, C3, C4 | 43.3, 20.1, |
| fibuligera BGLI | | | | 26.2, 27.1 |
| | | | | (as for |
| | | | | activity) |
| Saccharomycopsis | 18.9 | 0.47 | PNPG, C2, C3, C4 | 168, 0.8, |
| fibuligera BGLII | | | | 1.7, 1.5 |
| | | | | (as for |
| | | | | activity) |
| | NR | NR | 115 000 U/L, | NR |
| | | | ~12 800 U/g DCW (PNPG) | |
| | NR | NR | 112 U/g DCW (PNPG) | NR |
| | NR | NR | 19 U/g DCW (PNPG) | NR |
| Bacillus circulans BGL | NR | NR | 450 U/L, ~50 U/g DCW | NR |
| | | | (PNPG) | |
| Endomyces fibuliger | NR | NR | 2023 U/g DCW (C2) | NR |
| BGLI | | | | |
| | NR | NR | 172 U/g DCW (C2) | NR |
| Ruminecoccus | NR | NR | 5.46 U/g DCW (PNPG) | NR |
| flawefaciens CELI | | | | |
| Candida wickechamii | NR | NR | 0.298 U/L (PNPG) | NR |
| bglB | | | | |
| Bacillus polymyxa bglA | NR | NR | 2.3 U/mg total protein | NR |
| Candida molischiana | NR | NR | 48 U/L (PNPG) | NR |
| BGLN | | | | |
| Cellulomonas biazotea | NR | NR | 2000 U/L (C2) | NR |
| Beta-glucosidase | | | | |
| Trichoderma reesel bgl I | NR | NR | PNPG | NR |
| Bacillus circulans BGL | NR | NR | 64 U/g DCW (PNPG) | NR |
| Candida pelliculosa BGL | NR | NR | 17 500 U/L, | NR |
| | | | ~1950 U/g DCW (PNPG) | |
| Aspergillus niger BGL | NR | NR | Xglu | NR |
| Kluyveromyces | NR | NR | 1700 U/g total protein (C2) | NR |
| fragilis BGL | | | | |

U = micromole substrate released/min,
NR = not reported: italics indicate calculation based on assumptions (0.45 g DCW/g glucose, 0.45 g protein/g DCW, 1.5 × 10⁷ cells/mg DCW, 1 OD(600) = 0.57 g DCW/L).
CBH = cellobiohydrolase,
EG = endoglucanase,
BGL = beta-glucosidase,
AC = amorphous cellulose,
BMCC = bacterial microcrystalline cellulose,
BBG = barley beta-glucan,
CC = crystalline cellulose,
IOSC = insoluble cellooligosaccharides,
C2 = cellobiose,
C3 = cellotriose,
C4 = cellotetraose,
PNPC = p-nitrophenol cellobioside,
PNPL = p-nitrophenol lactoside,
MUC = methylumbelliferyl cellobioside,
MUL = methylumbelliferyl lactoside.
Xglu = 5-bromo-4-chloro-3-indolyl-β-n-glucopyranoside

TABLE 2
Hemicellulase components expressed in S. cerevisiae.

| | | | Substrate(s) activity was | |
|---|---|---|---|---|
| | | | detected against (values | Specific |
| Organism & | Titer | % cell | indicate activity measured | activity |
| gene/enzyme | (mg/L) | protein | per L culture broth) | (U/mg) |
| Xylan degradation: | | | | |
| β-Xylamase | | | | |
| Cryprococcus albidus | NR | NR | 1.3 U/mg protein (xylan) | NR |
| XLN | | | | |
| Aspergillus | NR | NR | 18000 U/L (BG-xylan) | NR |
| kawachill xynC | | | | |
| Trichoderma racsei xynZ | NR | NR | 72000 U/L (BG-xylan) | NR |
| | NR | NR | 51600 U/L (BG-xylan- | NR |
| | | | coexpression | |
| Aureobasidium | ~13.1 mg/L | 1.6% | 26200 U/L (BG-xylan) | 2000 U/mg |
| pullulans xynA | | | | (native) |
| β-Xylosidase | | | | |
| Trichoderma racsei bxlI | NR | NR | 19.6 U/L (PNP-β-X), xylan, | NR |
| | | | PNP-β-G, xylobiose | |
| Bacillus pumilus xynB | NR | NR | 3,4-U/L (PNP-β-X) | NR |
| Aspergillus niger xlnD | NR | NR | 318 U/L (PNP-β-X), | NR |
| | | | xylobiose, xylotriose | |
| Aspergillus oryzae xylA | NR | NR | 316 U/g DCW (PNP-β-X) | NR |
| α-Glucuranidase | | | | |
| Aureobasidium pullulans | 0.1 mg/L | 0.013% | 5 U/L (ABIU, ATRU, ATEU) | 135 U/mg |
| aguA | | | | (ATEU) |
| α-L-Arabinofuranosidase | | | | |
| Aspergillus niger abfB | NR | NR | 1400 U/L (PNPA) | NR |
| | 117.3 mg/L | 5.2% | 67.8 U/L (PNPA) | 5.78 U/mg |
| | NR | NR | 25.7 U/L (PNPA) | NR |
| Trichoderma racsei abf1 | NR | NR | 205 U/L (PNPA), | NR |
| | | | arabinosylan | |
| Mannan degradation: | | | | |
| α-Mannanase | | | | |
| Trichoderma racsei | 150 μg/L | NR | 132 U/L (LBG) | NR |
| man1 | | | | |
| Aspergillus aculeatus | 118 mg/L | 5.04% | 31280 U/L (LEG), INM | 82 U/mg |
| man1 | | | | |
| Orpinomyces PC-2 manA | 6 mg/L | 0.74% | 1150 U/L (LBG), INM | 179 U/mg |
| αGalactosidase | | | | |
| Trichoderma racsei agl1 | NR | NR | 516 U/L (PNPαGal) PNPA, | NR |
| | | | raffinose, melibiose, LBG, | |
| | | | PGGM | |
| Trichoderma racsei agl2 | NR | NR | 20.8 U/L (PNPαGal) | NR |
| | | | LEG, PGGM | |
| Trichoderma racsei agl3 | NR | NR | 1.32 U/L (PNPαGal) | NR |
| | | | LBG, PGGM | |
| Xyloglucan degradation: | | | | |
| Endo-β-1,4-glucanase | | | | |
| Aspergillus andeatus | NR | NR | AZCL XG | NR |
| α-Xylosidase | | | | |
| Arabidopsis thaliana | NR | NR | 0.0006 U/g wet weight | NR |
| AtXYL1 | | | (EG digested xyloglucan) | |

U = micromole substrate released/min,
DCW = dry cell weight,
NR = not reported; substrate used for activity determination is given in parentheses;
italics indicates calculation based on assumptions (0.45 g DCW/g glucose, 0.15 g protein/g DCW, 1.3 × 10⁷ cells/g DCW, 1 OD(600) = 0.57 g DCW/L).
BG-xylan = birchwood glucuronoxylan,
PNP-β-X = p-nitrophenyl-β-D-xylopyranoside,
AZCL-XG = azurine-dyed cross-linked xyloglucan,
ABIU = aldobiouronic acid,
ATRU = aldotriouronic acid,
ATEU = aldotetraouronic acid,
PNPA = p-nitrophenyl-α-L-arabino-furanoside,
LBG = locust bean gum,
INM = ivory nut mannan,
PGGM = pinewood galactoglucomannan,
PNPαGal = p-nitrophenyl-α-D-galactopyranoside

High concentrations of fermentable sugars include concentrations that are about 100 g/L to about 400 g/L. Thus, in some embodiments, the yeast (e.g., engineered yeast) is cultured in medium having a fermentable sugar concentration of at least 100 g/L. In some embodiments, the yeast is cultured in medium having a fermentable sugar concentration of about 100 g/L to about 400 g/L. For example, in some embodiments, the yeast is cultured in medium having a fermentable sugar concentration of 100 g/L, 150 g/L, 200 g/L, 250 g/L, 300 g/L, 350 g/L or 400 g/L.

Industrial fermentation processes may also include culturing yeast at a high cell density. Thus, in some embodiments, the yeast (e.g., engineered yeast) is cultured at a cell density of about 1×10⁶ to about 1×10⁹ viable cells/ml. For example, in some embodiments, the yeast is cultured at a cell density of about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸ or about 1×10⁹ viable cells/ml.

In some embodiments, the yeast (e.g., engineered yeast) is cultured at an optical cell density, measured at a wavelength of 600 nm, of about 1 to about 150 (i.e., OD₆₀₀ is about 1 to about 150). For example, in some embodiments, the OD₆₀₀ of a cell culture containing fermenting yeast cells is about 1, about 5, about 10, about 15, about 20, about 25, about 30, about 35, about 40, about 45, about 50, about 55, about 60, about 70, about 75, about 80, about 85, about 90, about 95, about 100, about 110, about 120, about 130, about 140, about 150. In some embodiments, the OD₆₀₀ of a cell culture containing fermenting yeast cells is about 20 to about 30.

In accordance with the present disclosure, the yeast (e.g., engineered yeast) may be cultured in standard synthetic complete medium with nutrient drop-out for selection when appropriate (Sherman, F. Meth Enzymol 350, 3-41 (2002), incorporated by reference herein). For example, yeast synthetic complete (YSC) medium may contain a nitrogen base without amino acids and ammonium sulfate (e.g., BD-Difco Yeast Nitrogen Base catalog #233520) with or without nutrients. In some embodiments, the culture medium is adjusted for K⁺, H⁺ and/or Na⁺ concentration.

The present disclosure also provides methods of ethanol production that comprise culturing yeast cells in culture medium (e.g., complex media such as the media described in Example 9) that comprises fermentable feedstock and a potassium salt selected from potassium phosphate monobasic (KH₂PO₄ or K-Pi), potassium phosphate dibasic (K₂HPO₄) and potassium sulfate (K₂SO₄).

The potassium salt may be present in the culture medium in an amount sufficient to produce at least 100 g/L, or at least 150 g/L ethanol. In some embodiments, the potassium salt is in an amount sufficient to produce about 100 g/L to about 300 g/L of ethanol. For example, in some embodiments, the potassium salt is in an amount sufficient to produce about 100 g/L, about 150 g/L, about 200 g/L, about 250 g/L or about 300 g/L.

In some embodiments, the culture medium further comprises potassium hydroxide (KOH), which is present in an amount sufficient to maintain, in the culture medium, a pH of at least 3. Thus, in some embodiments, KOH may be used to adjust the pH of culture medium comprising a potassium salt such as, for example, KCl. In some embodiments, KOH is used to adjust the pH of the culture medium to about 3, about 3.5, about 4, about 4.5, about 5, about 5.5, about 6, about 6.5, about 7, about 7.5 or about 8. In some embodiments, the pH of culture medium (e.g., containing KCl) is adjusted or maintained at a pH within a range of 3 to 8 or about 3 to about 8 (e.g., a pH of 3, 3.5, 4, 4.5, 5, 5.5, 6, 6.5, 7, 7.5 or 8).

The concentration of potassium salt in the culture medium may be about 15 mM to about 100 mM. For example, in some embodiments, the concentration of potassium salt in the culture medium is about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM. In some embodiments, the concentration of potassium salt in the culture medium is about 25 to about 50 mM, about 35 to about 65 mM, or about 50 mM to about 75 mM.

Industrial fermentation processes may also include culturing yeast at elevated temperatures (e.g., 30° C. to 70° C., or higher). Typically, alcohol production decreases when yeast cells are cultured at elevated temperatures (e.g., greater than 25° C.). This is particularly problematic for fermentations in warm climates (e.g., summer months). Surprisingly, the results from experiments described herein demonstrate that elevated K⁺ and pH confer cellular resistance to the adverse effects (e.g., decreased ethanol production) of heat. As shown in Example 12, the addition of KCl and KOH to fermentations improved ethanol production by ˜50% at 37° C. and by ˜16% at 45° C. Thus, the present disclosure contemplates culturing yeast cells (e.g., unmodified or modified) at a temperature of 30° C. to 70° C. (e.g., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C. or higher) in culture medium that comprises fermentable feedstock and a potassium salt.

### EXAMPLES

### Example 1

### Potassium Phosphate (K—Pᵢ) Boosts Ethanol Production by Enhancing Tolerance

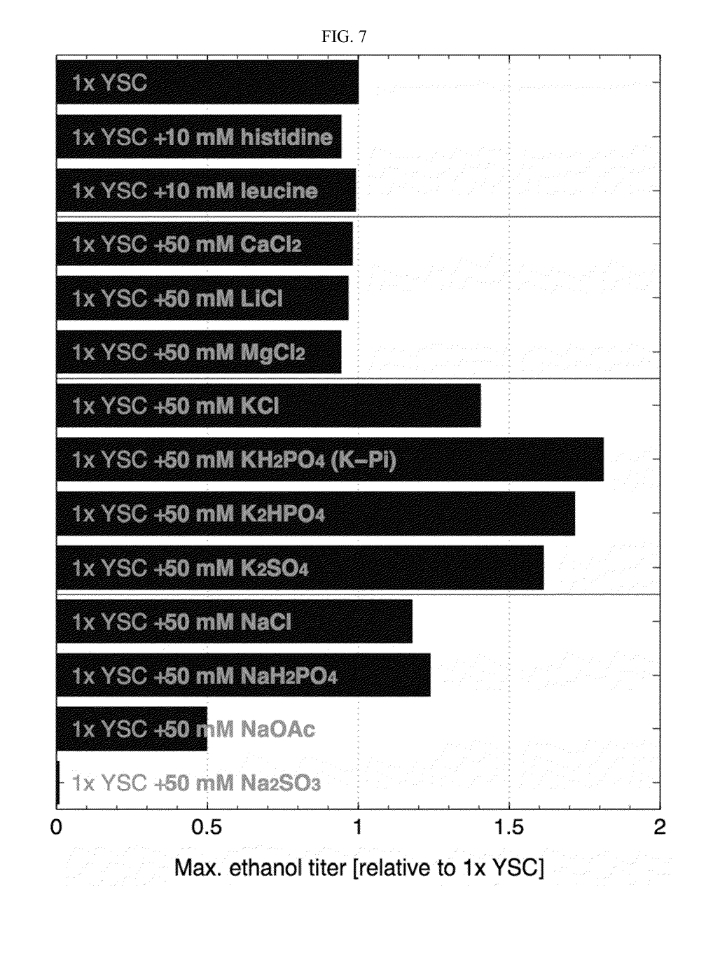

To investigate the possibility that ethanol disrupts the integrity of the plasma membrane and that altering the ionic composition of the fermentation medium could provide stability and improve ethanol performance, ethanol production was measured from a laboratory strain (S288C) cultured under high cell density (initial OD₆₀₀ 20-30) and high glucose (300 g/L) conditions supplemented with a variety of additives to standard synthetic medium (1×YSC). The addition of 50 mM (mono)potassium phosphate (K—Pᵢ), or potassium phosphate monobasic (KH₂PO₄), induced the largest improvement, raising output by >50% (FIG. 7). Over the course of a 4-day culture, elevated K—Pᵢ enhances ethanol titer and productivity, two key characteristics of fermentative performance (FIG. 1A). The final ethanol titer of ˜140 g/L was unexpectedly high given the general underperformance of most inbred laboratory strains and known low ethanol tolerance of the S288C genetic background^{5,11,12}.

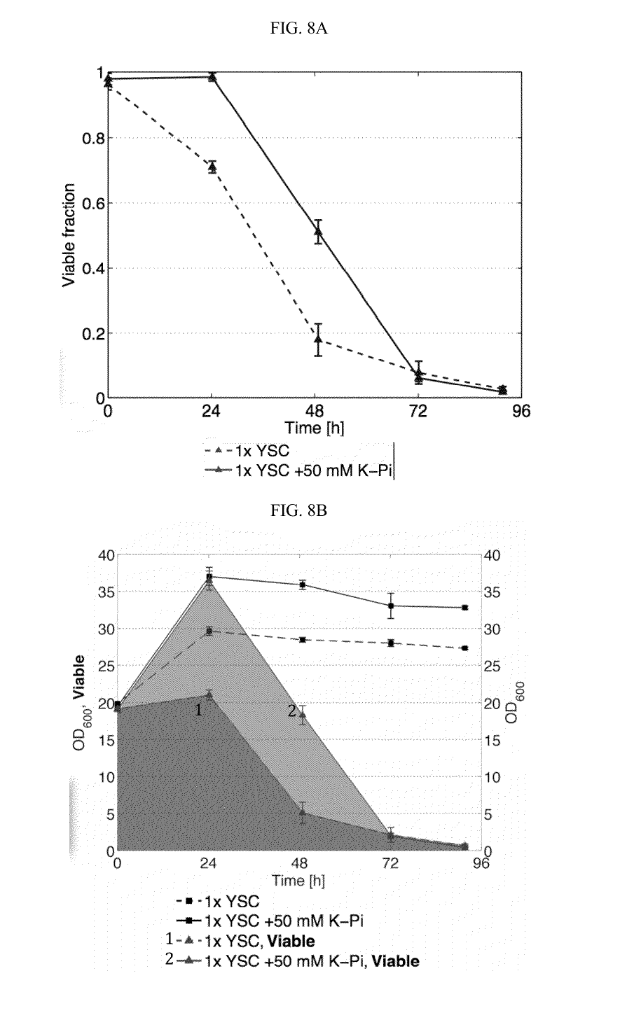

A comparison of cell densities and ethanol titers during fermentation revealed that K—Pᵢ supplementation enhances ethanol tolerance: the ˜25% additional yeast biomass arising from high K—Pᵢ was insufficient to account for the >50% rise in ethanol output (FIG. 1B). In particular, the underlying population of viable cells in elevated K—Pᵢ was disproportionately greater despite the increased toxicity imposed by higher ethanol production (FIGS. 1B, 8A). Specific ethanol productivity (e.g., rate of ethanol increase normalized by the corresponding mean live population) remained unchanged, demonstrating that elevated K—Pᵢ exerts its effect, not on per-cell output, but by boosting tolerance and the total quantity of live cells (FIG. 1A). Furthermore, because this fraction is actively fermenting, final product titers will be governed both by the greater number of live cells and the length of time that such increased viability can be maintained against rising ethanol toxicity. Thus, the time integral of the viable biomass fraction is the main determinant of ethanol output and, as a function of tolerance, the primary variable enhanced by K—Pᵢ supplementation (FIG. 8B, 3B, 3C).

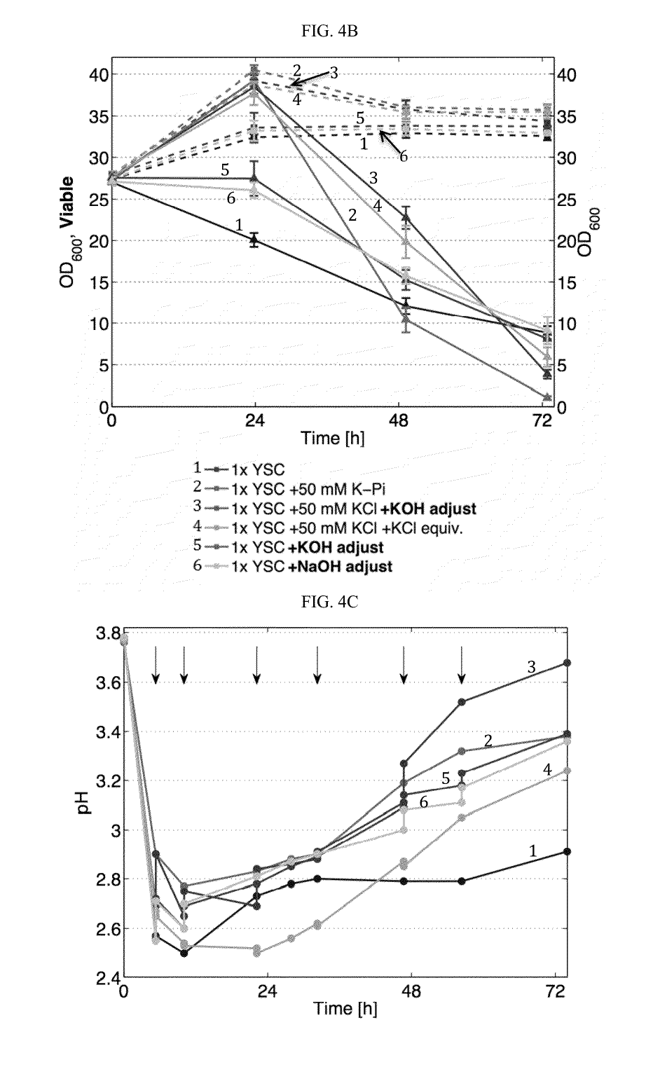

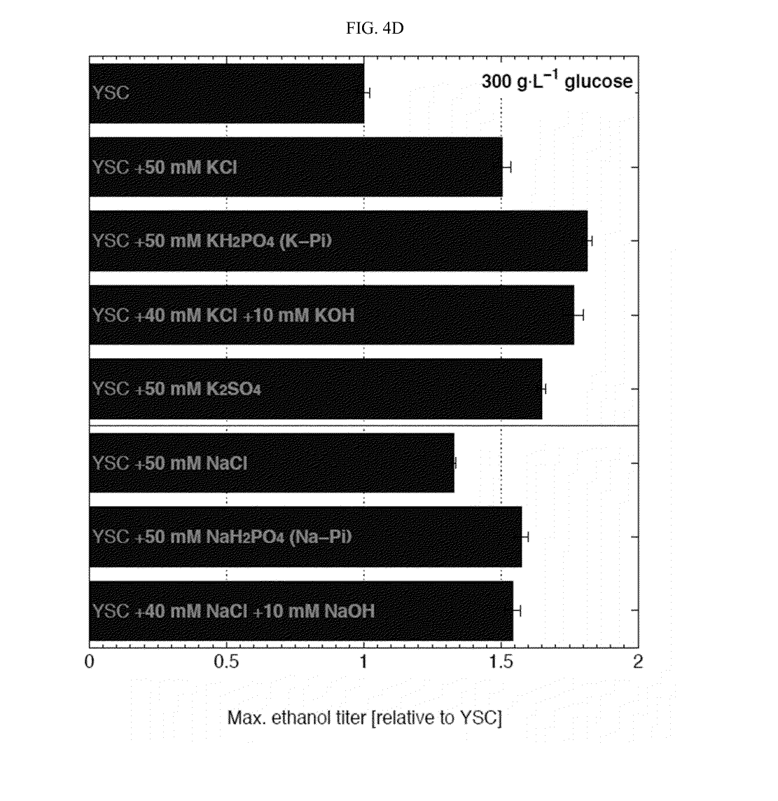

Monopotassium phosphate (K-Pi) added to standard yeast synthetic complete (YSC) medium induced the greatest improvement (FIG. 7), an effect that was dissected into components deriving from elevated potassium (K+) and pH. Specifically, when the pH of cultures containing elevated potassium chloride (KCl) was manually adjusted with potassium hydroxide (KOH) throughout the course of fermentation to match that of cultures containing elevated K-Pi, ethanol titers were statistically indistinguishable (p=0.09 from two sample t-test; p≦7.6≦10-3 for other pairs) from one another (FIG. 4D). It was also determined that KCl elicited a statistically higher improvement than sodium chloride (NaCl), and that supplementation with NaCl and sodium hydroxide, or with monosodium phosphate, demonstrated a distinguishable boost over NaCl alone (p≦2×10⁻⁴ from pair-wise t-tests). Thus, the greatest improvements in ethanol production derive specifically from the increase in K+ concentration and reduction in acidity of the fermentation medium.

Over the course of a 3-day culture, supplementation with KCl and KOH enhanced ethanol titer and volumetric productivity (grams of ethanol per volume per hour), two key benchmarks of fermentative performance (FIG. 1C). Additionally, compared with equimolar KCl or matched pH alone, the combination of K⁺ supplementation and acidity reduction enabled the complete utilization of glucose and decreases in the synthesis of acetic acid and glycerol, two undesired byproducts of fermentation. The 128±0.7 g/L (SD) concentrations observed herein were achieved using a purely synthetic formulation, allowing for the identification and precise control of environmental components that impact ethanol tolerance.

The boost in ethanol production from KCl and KOH supplementation did not arise simply from an increase in cell number, but from an increase in cell tolerance. Specifically, the 80±1.3% (SD) jump in titer (FIG. 1C) was accompanied only by an 11±4.6% (SD) average higher cell density (FIG. 1D); therefore, cell growth alone could not explain the rise in output. This discrepancy, however, was resolved when fractions of cells remaining alive throughout fermentation were directly assessed, and it was discovered that the addition of KCl and KOH enhanced overall population viability (FIG. 1D). This enhancement, furthermore, occurred despite the increase in toxicity imposed by higher accumulations of ethanol.

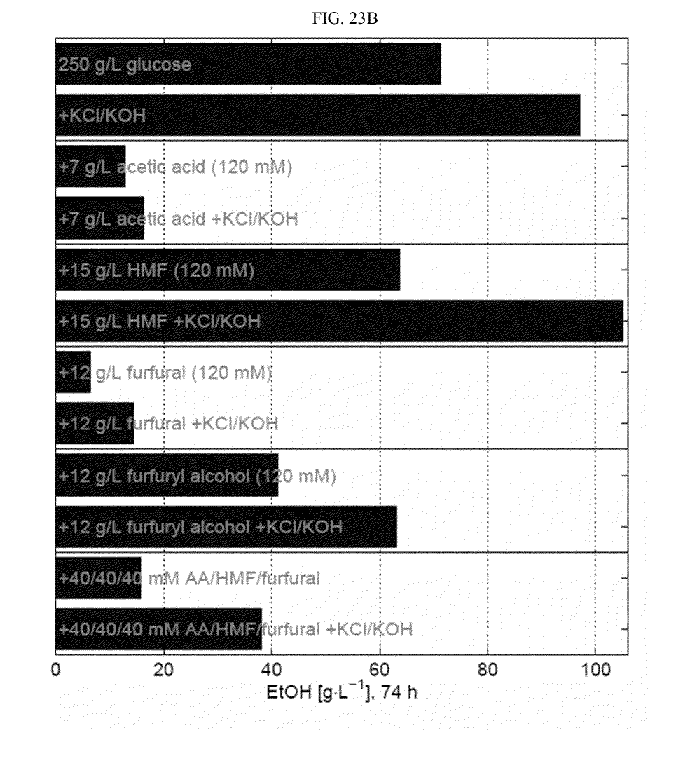

When specific productivities were calculated—rates of ethanol increase normalized by the live, rather than total, cell population—the values from KCl and KOH supplementation differed from the control by an average of 11±7.5% (SD) (FIG. 1C). That these differences account for a minor portion of the increase in titer suggests that elevated K⁺ and pH acts primarily not by affecting per-cell output, but by boosting tolerance and the overall viable cell population. Additionally, these effects are observed fully in fermentations conducted in anaerobic bioreactors, demonstrating that these tolerance improvements do not depend on oxygen availability and can scale to higher-volume environments (FIGS. 23B and 23C).

### Example 2

### K—Pᵢ Enhances Tolerance to Alcohol Shocks in High Glucose

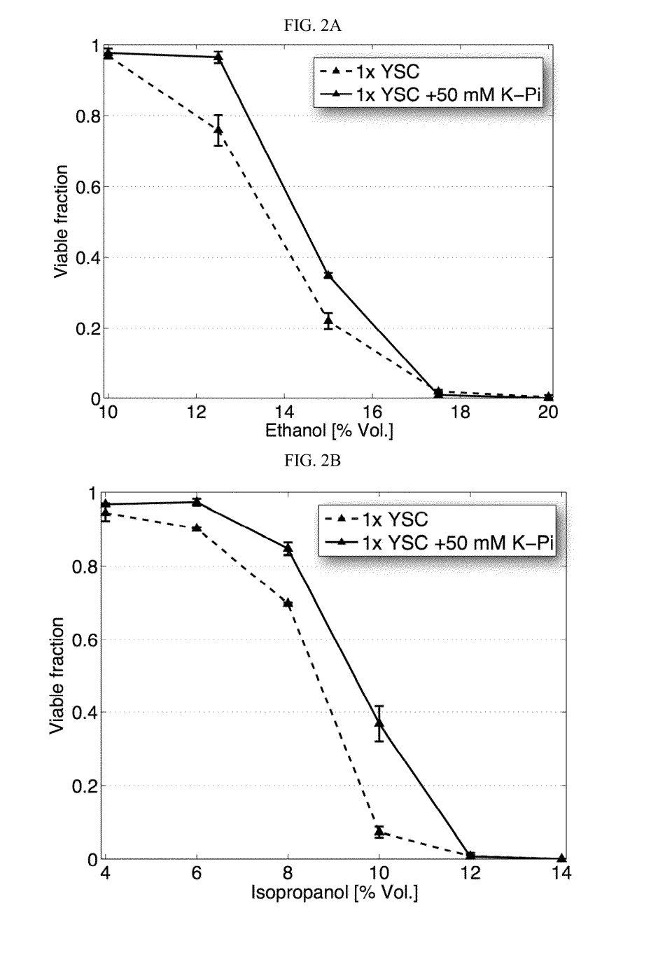

To isolate specifically the impact of K—Pᵢ on tolerance under extreme sugar and ethanol conditions, the ability of yeast to withstand artificial steps in ethanol concentration against a background of 300 g/L glucose was quantified. When cells growing in elevated K—Pᵢ were transferred to identical medium containing 10-20% ethanol and viability assayed after several hours, survival was indeed enhanced over cells treated in unmodified medium (FIG. 2A). This indicated that the impact of high K—Pᵢ was immediate, did not require adaptation to ethanol over the course of days, and overcame the combined toxicity of high glucose and ethanol.

Furthermore, the boost in tolerance conferred by elevated K—Pᵢ extended beyond ethanol. When the shock assay was repeated using steps of isopropanol, the viability was similarly enhanced among cells cultured with K—Pᵢ supplementation (FIG. 2B). That the improvements were not ethanol specific suggests that heightened K—Pᵢ may augment a more general cellular process involved in alcohol resistance or membrane integrity.

### Example 3

### The Effects of High K—Pᵢ are Independent of Osmotic Shock, Phosphate Starvation or Nutrient Starvation

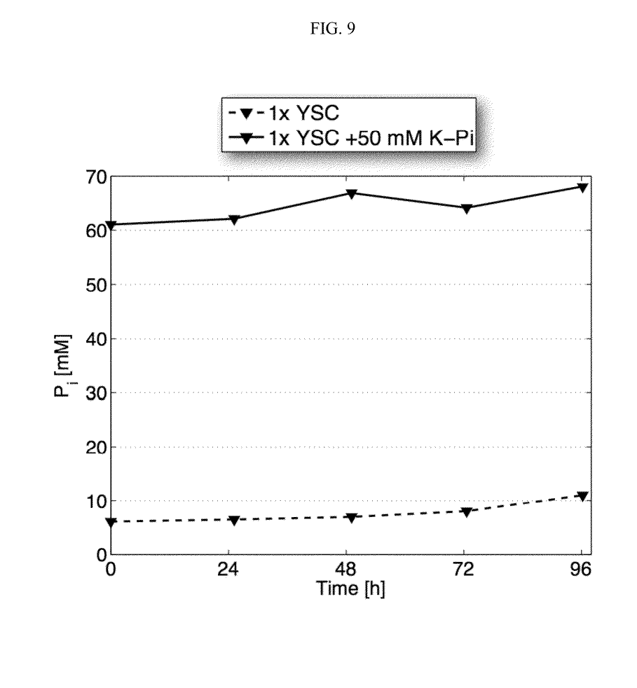

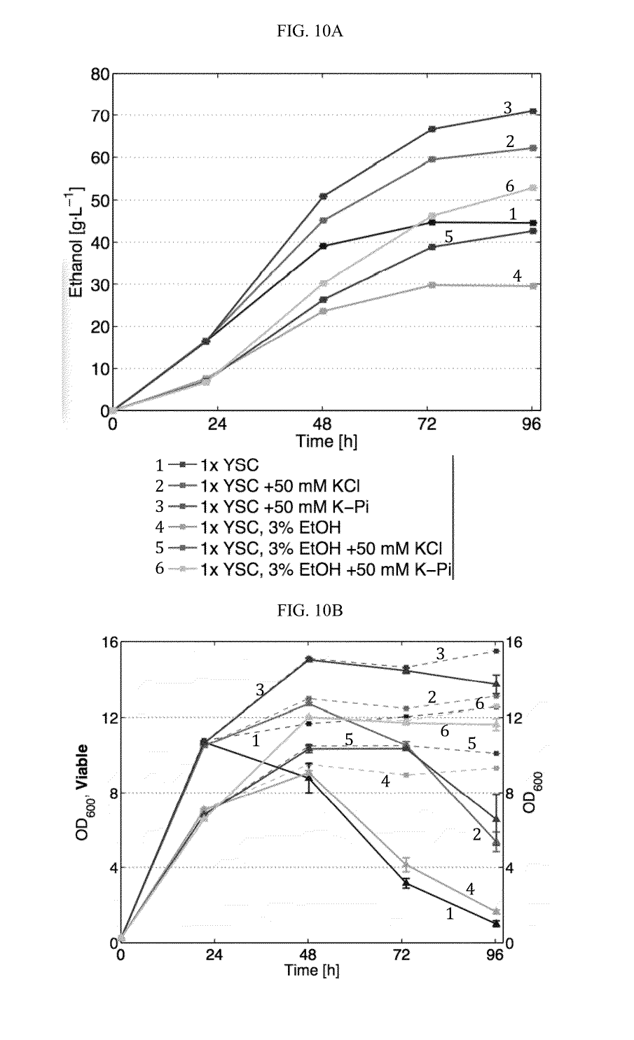

A number of explanations for the effects of high K—Pᵢ such as osmotic shock, phosphate starvation, and nutrient starvation were ruled out. First, the concentration of K—Pᵢ used herein was below the threshold that has been reported to trigger K⁺-mediated salt shock^{13,14}. Second, it is possible that phosphate may become depleted at high cell density despite studies demonstrating the amount of phosphate in standard synthetic medium (˜7 mM) is in excess at low cell density (OD₆₀₀<1)¹⁵. However, direct measurement showed that, even at high cell density (where growth is <2 fold), phosphate concentrations remained unchanged (FIG. 1B, FIG. 9). Third, to address the possibility that cells may have depleted various non-phosphate nutrients, cultures were inoculated to a cell density several hundred fold lower (starting OD₆₀₀≈0.1-0.2). These conditions were also repeated with the addition of 3% ethanol to impose a mild ethanol stress at the start of fermentation. In both scenarios, nutrients remained in abundance, yet improved viability and ethanol production with K—Pᵢ supplementation was still observed (FIGS. 10A, 10B). Thus, elevated K—Pᵢ likely participates in a process specific to alcohol tolerance and does not simply alleviate nutritional constraints created by high cell density.

### Example 4

### The Effects of High K—Pᵢ are Independent of Cellular Phosphate Homeostasis

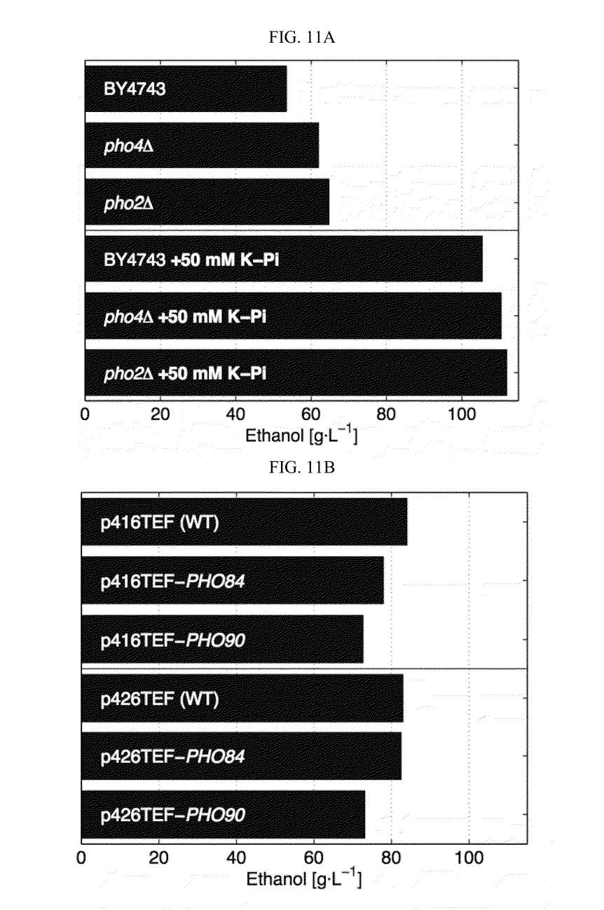

Perturbations to intracellular phosphate regulation also did not impact the improvements conferred by supplemental K—Pᵢ. Strains defective in responding to environmental phosphate starvation or abundance (pho4Δ or pho2Δ) demonstrated enhanced ethanol output in high K—Pᵢ that was indistinguishable from wild-type (FIG. 11A). Furthermore, it is unlikely that K—Pᵢ supplementation generates its improvements by raising intracellular concentrations of phosphate. Internal phosphate and rates of uptake are likely to already be saturating in unmodified medium as ˜7 mM K—Pᵢ is significantly above the Kₘ of all known plasma membrane phosphate transporters¹⁶. Moreover, overexpression of either the high affinity transporter PHO84 or the low affinity transporter PHO90 to force cytosolic phosphate to super-physiological levels failed to increase ethanol titers (FIG. 11B). That intracellular phosphate is likely to already be at a physiological maximum in unaltered medium, combined with prior observations showing that inorganic phosphate levels remain unchanged during fermentation, suggest that elevated K—Pᵢ exerts its enhancing effects in an extracellular capacity.

### Example 5

### KCl Elicits Dose-Dependent Improvements on Ethanol Tolerance and Production

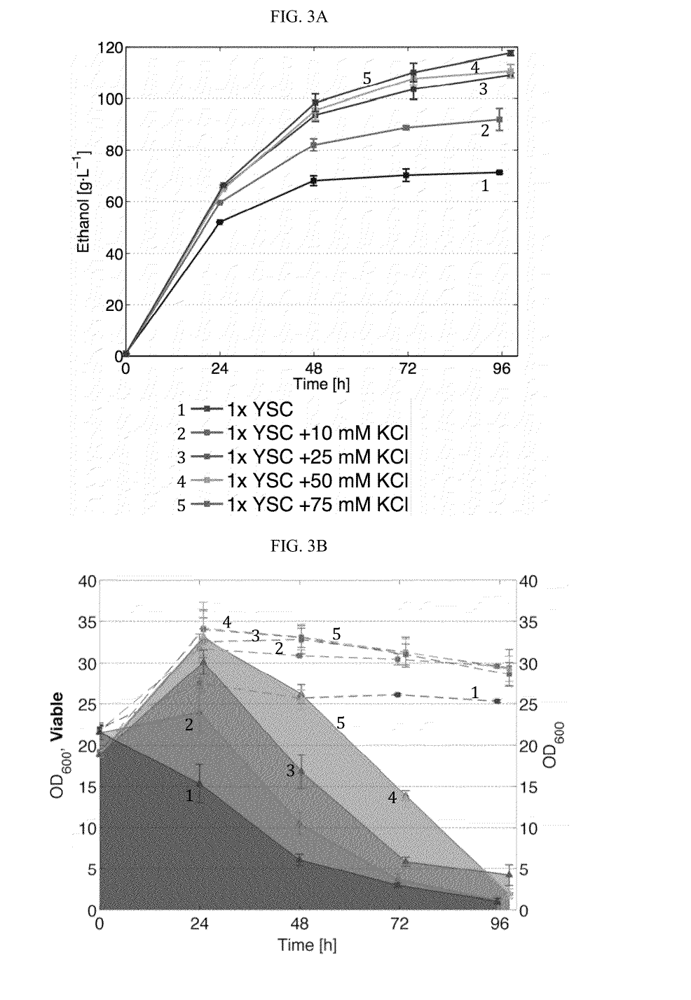

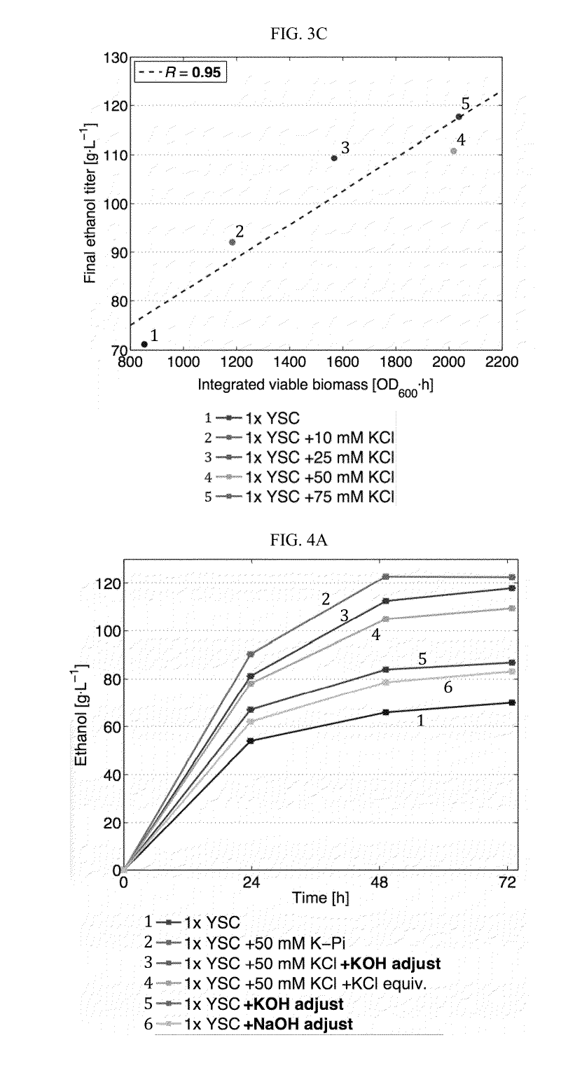

In dissecting the individual contributions of potassium versus phosphate, it was discovered that cationic potassium and anionic inorganic phosphate have separable, and quantitatively different, impacts on ethanol performance. Among the additives initially screened for effects on ethanol output, KCl had produced the largest enhancement within the panel of chloride salts tested (FIG. 7). When the amount of KCl added to synthetic medium was varied, there were dose-dependent improvements to ethanol output and viability during fermentation, suggesting that K⁺ wields a potentially univariate influence on ethanol tolerance (FIGS. 3A, 3B, 3C). Within the anionic additives, inorganic phosphate had generated the largest improvement (e.g., Na—Pᵢ improves ethanol titer over NaCl), confirming its independent, albeit smaller, influence on augmenting fermentation (FIG. 7).

### Example 6

### Potassium Supplementation and Acidity Reduction Recapitulate the Enhancements Conferred by K—Pᵢ

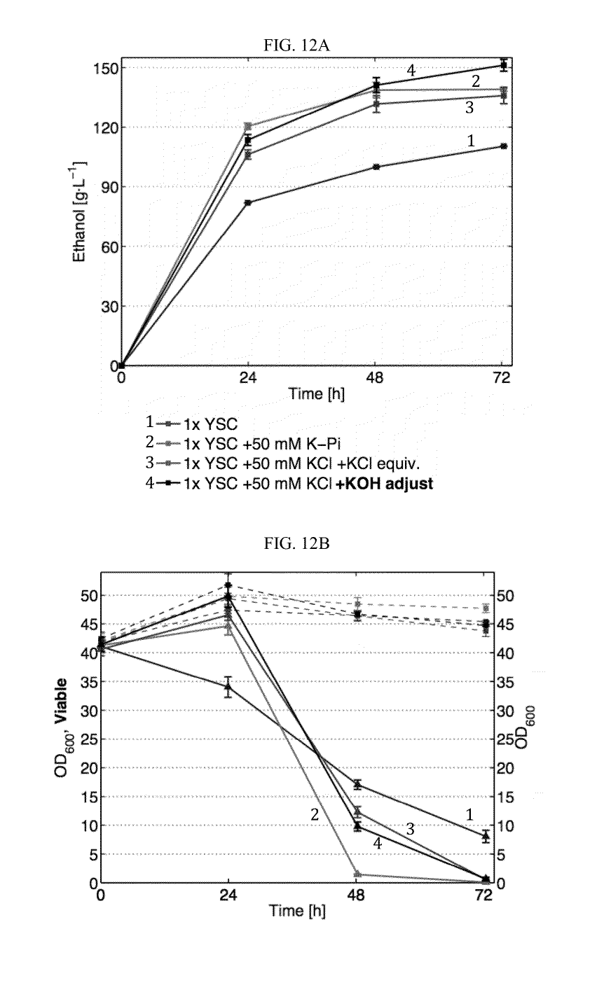

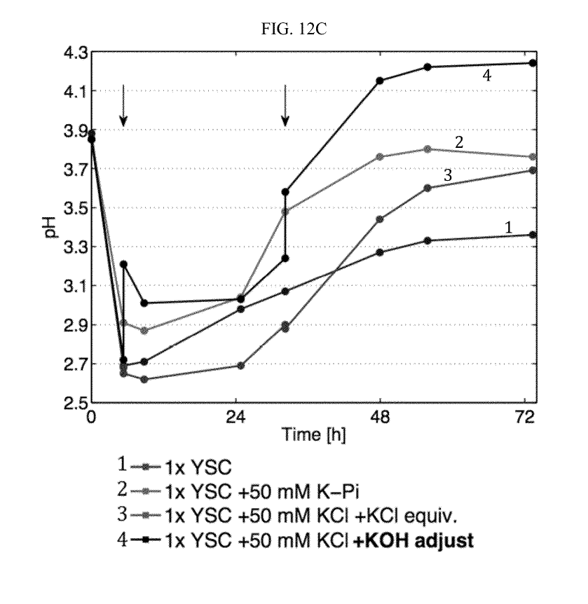

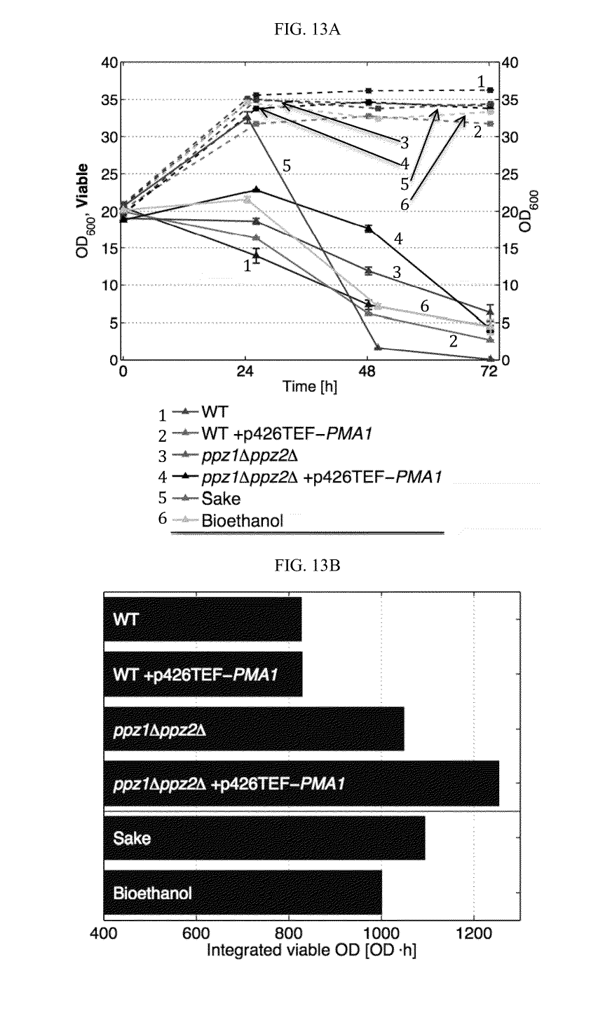

Supplementation of synthetic medium with KCl and manual adjustment of pH during the course of fermentation (using KOH to approximate that provided by elevated K—Pᵢ) achieved viability and ethanol production levels within 5% of those elicited by high K—Pᵢ (FIGS. 4A, 4B). Viability and ethanol production from KCl+KOH supplementation actually surpassed those conferred by K—Pᵢ supplementation when fermentations were inoculated with even higher cell densities (FIGS. 12A-12C). Here, however, the acidity of the medium varied in a range between the phosphate ion's two lower pKₐ's; therefore, its effect is not that of a buffer. Rather, the time course of acidity indicates that phosphate serves to draw pH strongly upward toward neutrality (FIG. 4C). Moreover, consistent with the quantitatively larger improvement exerted by cationic potassium over anionic phosphate, adjusting pH alone without supplemental KCl, using either KOH or NaOH, did not elicit similarly large enhancements. These data suggest that K⁺ may serve a more principal role than pH in controlling tolerance.

### Example 7

### Genetic or Culture Modifications Modulating the Potassium and Proton Gradients Elicit Corresponding Effects to Ethanol Production or Alcohol Tolerance

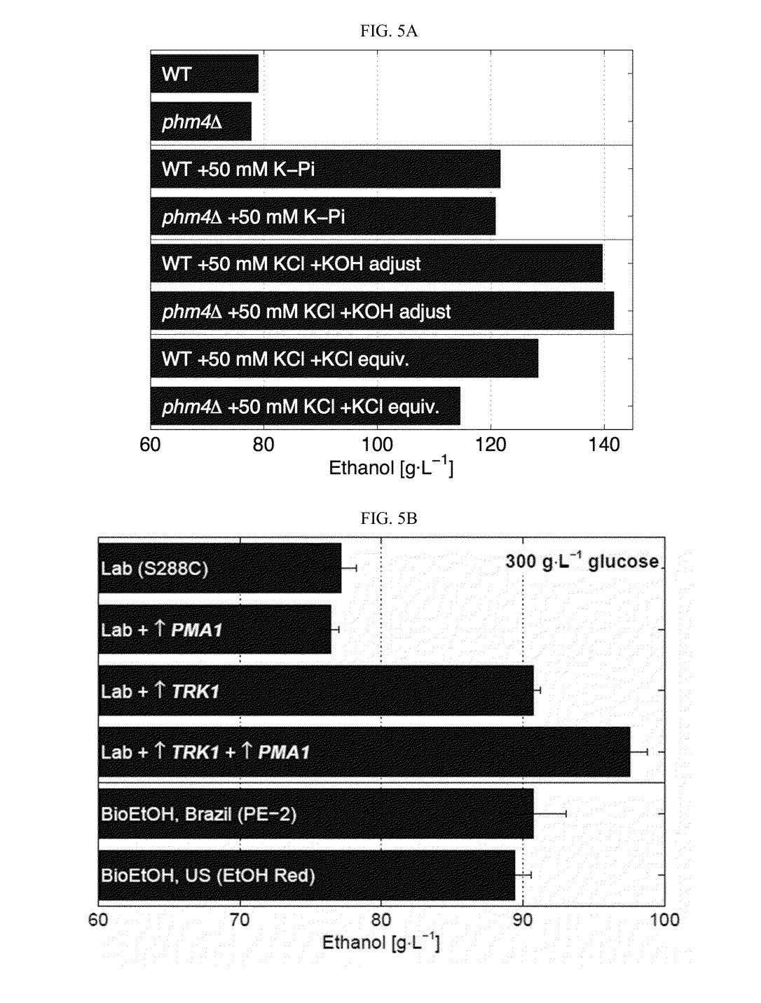

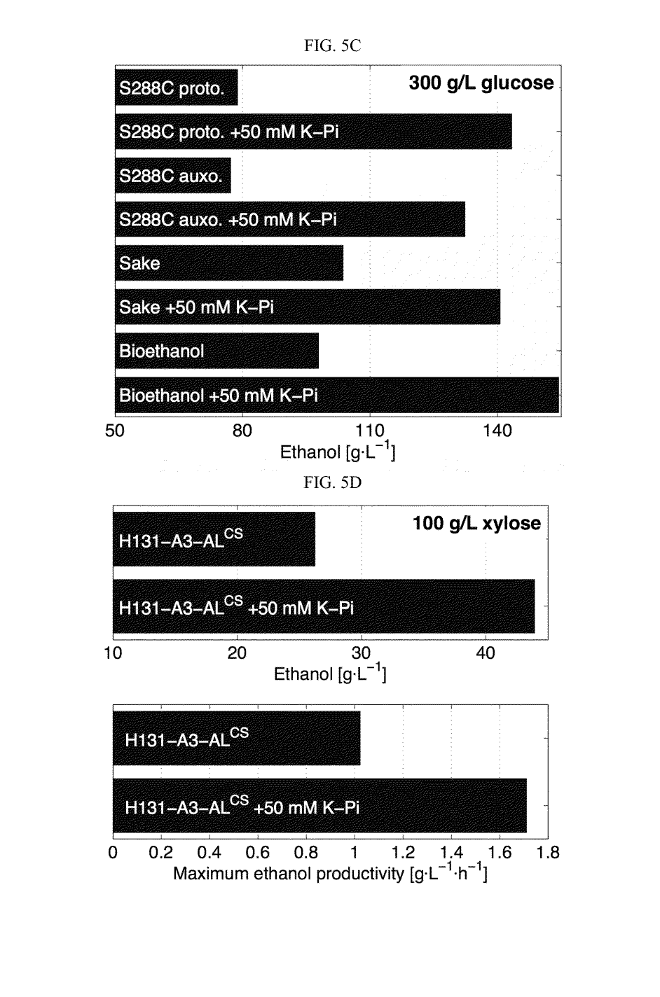

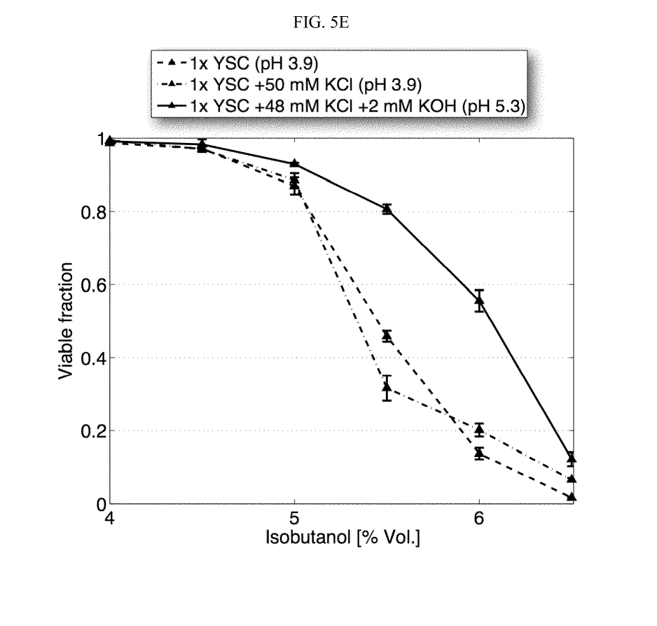

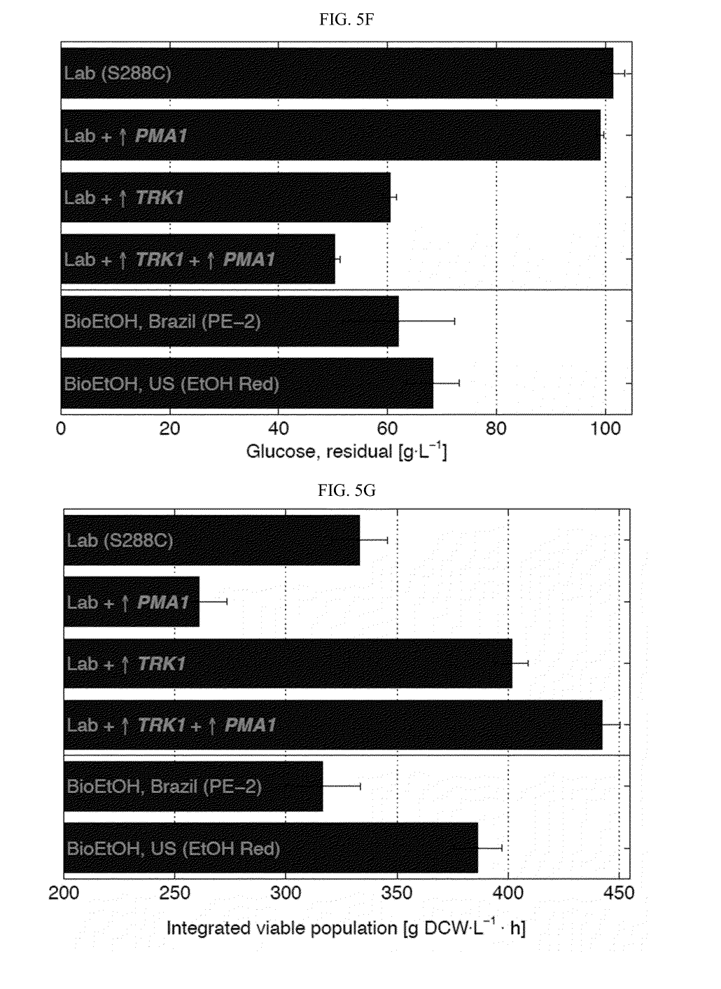

Genetic modifications to the ATP-driven K⁺ transporter TRK1 or H⁺ transporter PMA1 that specifically perturb or strengthen the opposing K⁺ and H⁺ electrochemical membrane gradients produced a corresponding impact on ethanol performance. As deletion of either of these gradient-establishing pumps affects viability, the H⁺ gradient was perturbed by reducing Pma1 protein levels while sustaining the K⁺ gradient by supplementation with KCl^{17,18}. A strain deleted for PHM4/VTC1, which is partially defective in Pma1 expression, exhibited a subdued ethanol boost when compared to wild-type¹⁹ (FIG. 5A). However, this defect was abolished, and the ethanol enhancement restored to wild-type levels, when the H⁺ gradient was assisted alongside that of K⁺ by supplementation with KCl and KOH, or K—Pᵢ, alternatively.

Furthermore, ethanol tolerance and production in unaltered medium was increased by the modification of just several genes by biologically augmenting the K⁺ and H⁺ gradients. Simultaneous deletion of the phosphatases PPZ1 and PPZ2 have been reported to result in hyperactivation of TRK1; due to the electroneutral co-dependence of the K⁺ and H⁺ gradients, the increase in K⁺ influx results in an emergent phenotype of elevated cellular resistance to low pH^{20,21}. Consistent with these enhanced gradients, the ppz1Δppz2Δ deletion strain exhibited a 18% improvement in ethanol titer (and correspondingly, productivity) over wild-type after 3 days of fermentation (FIG. 5B). When additional assistance to the H⁺ gradient was provided by overexpression of PMA1 in the ppz1Δppz2Δ background, ethanol titer was increased further to 27% over wild-type. These improvements mirrored enhancements in population viability, affirming the coupled nature of tolerance and production (FIGS. 12A, 12B). Overexpression of PMA1 without hyperactivation of TRK1 did not generate an enhanced ethanol phenotype, consistent with the only minor improvements seen in fermentations conducted solely with pH adjustment (FIGS. 5B, 4A). These results support the proposed notion that K⁺ uptake creates the dominant electromotive force and H⁺ efflux acts primarily as a response current²¹.

The control of electrochemical gradients is likely relevant to the production of ethanol from industrial strains. Genetic augmentation of the K⁺ and H⁺ gradients raised output of the laboratory strain to those matching or surpassing two ethanol tolerant commercial strains used in the production of sake wine and bioethanol^{12,22} (FIG. 5B). These modifications are, therefore, sufficient to create a superior phenotype previously available only through selection. Moreover, these industrial strains responded to K—Pᵢ supplementation which, as in the laboratory prototroph and auxotroph, enhances productivity (data not shown) and boosts ethanol output to titers near the molar conversion limit of ˜150 g/L (FIG. 5C). That both laboratory and industrial strains are inherently capable of producing titers far exceeding 100 g/L indicates that physically driven gradient assistance can supersede advantages conferred by genetic background and establishes tolerance as the primary bottleneck to performance.

Furthermore, altering the medium to augment membrane gradients enhanced ethanol production not only from glucose, but from xylose, an abundant carbon source from lignocellulosic feedstocks that cannot be metabolized by unmodified S. cerevisiae²³. Therefore K—Pᵢ supplementation was tested on H131-A3-AL^{CS}, a strain incorporating the Piromyces XYLA isomerase and P. stipitis XYL3 xylulokinase, and optimized to consume xylose²⁴. In high cell density, high xylose-only (100 g/L) fermentations, increases of ˜70% in both ethanol titer and productivity were found (FIG. 5D). Thus, physically strengthened membrane gradients enhanced ethanol performance in a strain-independent manner and enabled the near-comprehensive fermentation of sugars derived from cellulosic biomass²⁵.

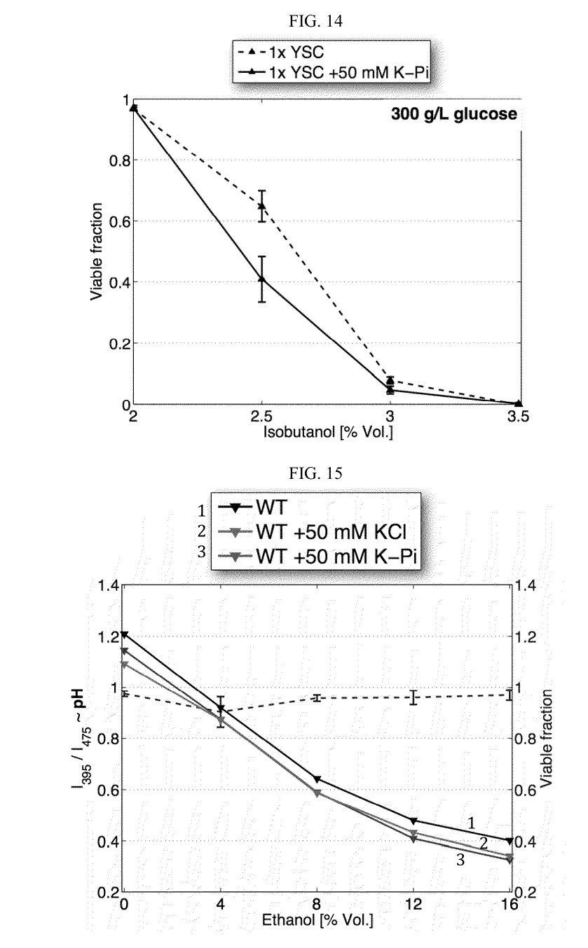

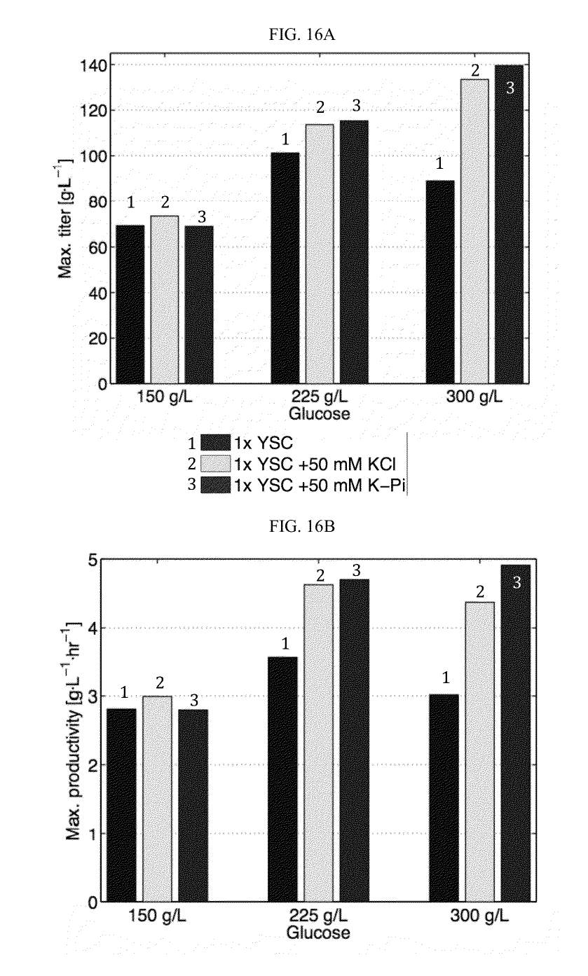

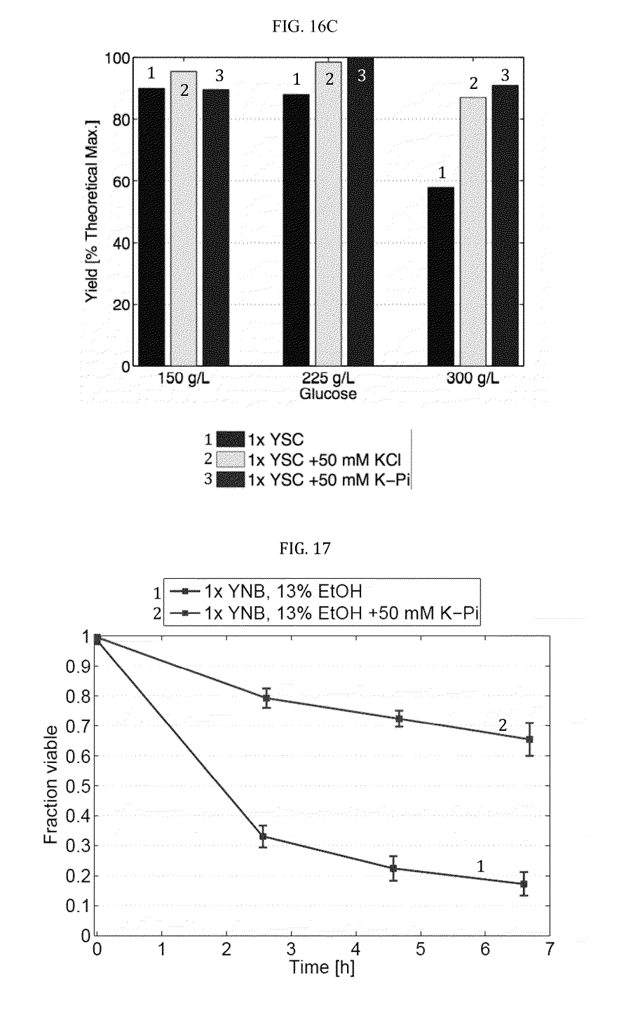

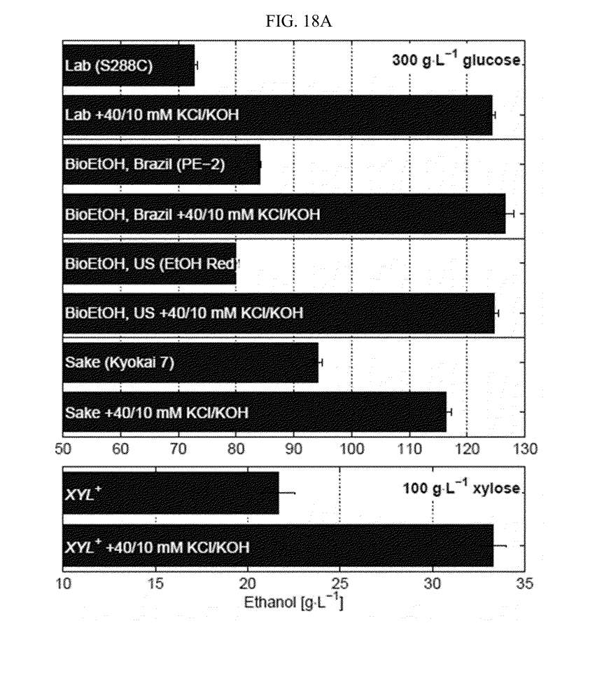

Elevated K⁺ and reduced acidity also evoke enhanced resistance to isobutanol, which has received much research attention as a strain engineering target despite its high toxicity to microbes²⁶⁻²⁸. When viability to steps of isobutanol in medium containing 300 g/L glucose was quantified (akin to the ethanol and isopropanol assays of FIG. 2), a reduced rate of survival triggered by elevated K—Pᵢ was observed (FIG. 14). However, due to the high concentration of glucose and the fact that supplemental K—Pᵢ promotes fermentation, it was surmised that the higher quantities of newly produced ethanol were approaching those of the added isobutanol and, thus, exacerbating toxicity. Therefore, to mitigate the contribution of ethanol, the assay was repeated in medium containing 5 g/L glucose. Given the greater potency of isobutanol compared to ethanol, additional KCl alone was insufficient to improve viability. However, consistent with the coupled nature of the K⁺ and H⁺ gradients, supplementation with K⁺, combined with a pH higher than any used previously (5.3 vs. 3.9) was, in fact, capable of enhancing resistance to isobutanol (FIG. 5E).

### Example 8

### Potassium Supplementation and Acidity Reduction Enhance Alcohol Tolerance by Strengthening the Potassium and Proton Electrogenic Gradients

The Examples provides a potential biophysical mechanism enabling elevated extracellular potassium and pH to counteract rising alcohol toxicity (e.g., during ethanol fermentation). FIG. 6 shows that in the absence of alcohol (top row), the opposing potassium (K⁺) and proton (H⁺) pumps maximally maintain the steep gradients of K⁺ and H⁺ that generate a major component of the homeostatic membrane potential. Rising alcohol levels permeabilized the plasma membrane and increase the leakage of ions that dissipate these gradients (middle row). Elevated potassium and pH, however, bolstered the gradients by slowing the rates of ion leakage (due to the reduced transmembrane K⁺ and H⁺ concentration differences) and allowing the cognate transporters to pump against a less precipitous differential (middle right). Thus, the threshold alcohol concentration that would otherwise destroy the separation of ions was raised, allowing cells to maintain viability at higher toxicity levels (bottom row).

### Example 9

### Chemically Undefined (“Complex”) Medium

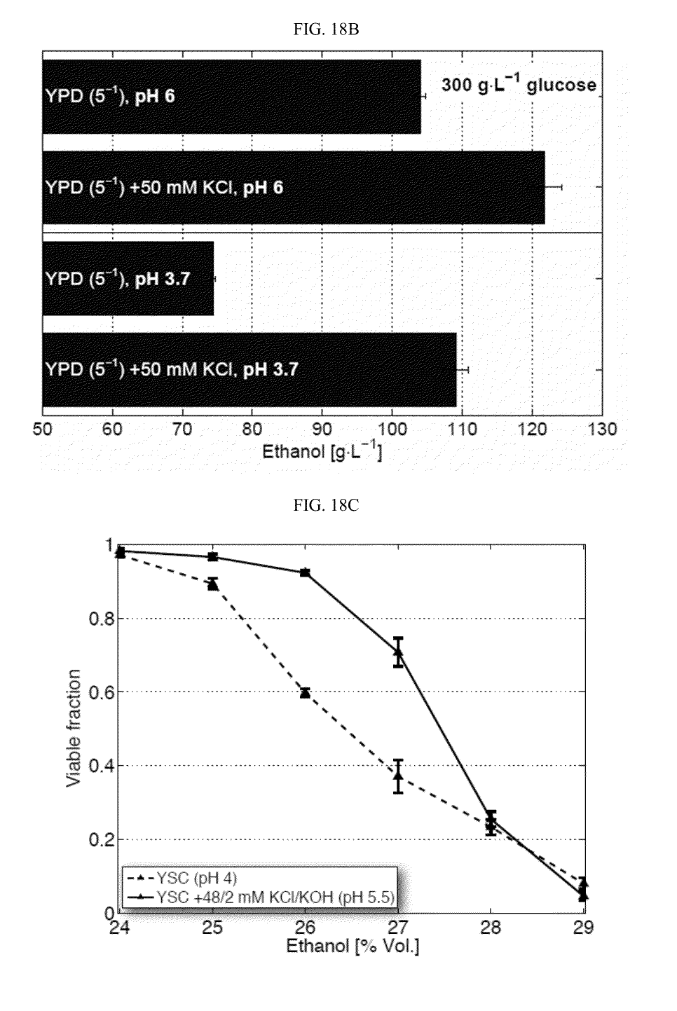

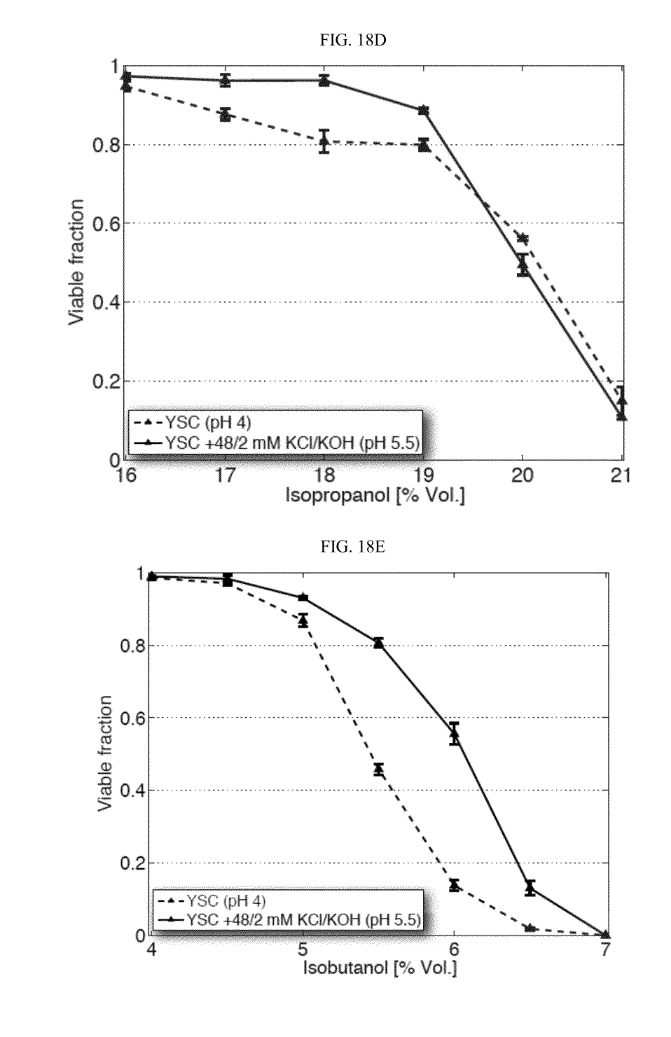

The enhancements conferred by elevated K⁺ and reduced acidity transcend genetic background and are elicited universally among a random sampling of industrial yeast strains. Those used in the production of biofuel ethanol in Brazil (PE-2) and the United States (Lasaffre Ethanol Red), and of sake wine in Japan (Kyokai No. 7), are typically the result of genetic selection efforts designed to isolate superior ethanol phenotypes. Consequently, all demonstrate distinctly higher ethanol output than laboratory strain S288C (10±1%-30±1.2% (SD)) when grown in unmodified medium (FIG. 18A). However, when subjected to KCl and KOH supplementation, all strains responded with enhancements in tolerance that enabled the complete consumption of glucose and titers of 116±0.9-127±1.6 g/L (SD). Under these conditions, S288C performed indistinguishably from the two industrial bioethanol strains (p≧0.08 from pair-wise t-tests). Thus, a strain traditionally deemed ethanol sensitive is capable—without genetic modification—of superior tolerance, indicating that K⁺ supplementation and acidity reduction drive a process that can supersede advantages conferred by genetic adaptation.

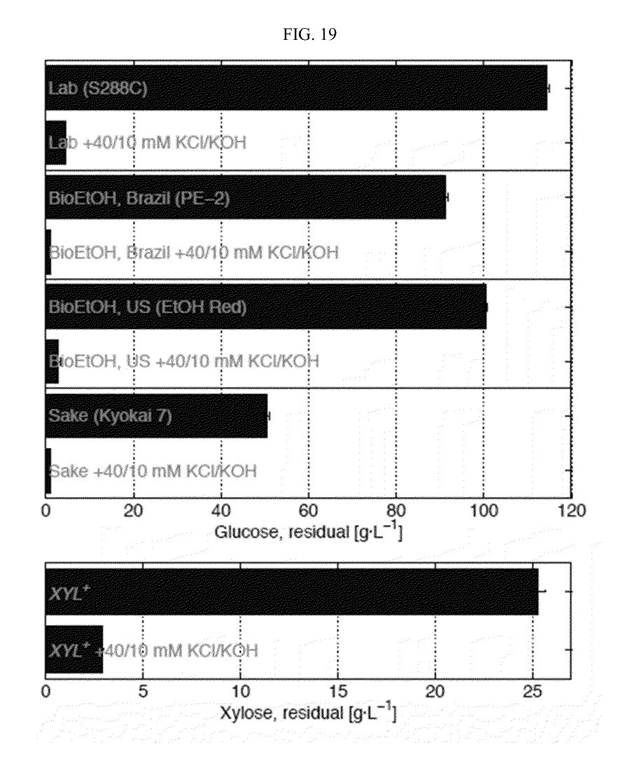

These adjustments to the medium, furthermore, enhance fermentation from xylose, an important hemicellulosic sugar that cannot be consumed by standard strains of S. cerevisiae. In an engineered strain, 22±0.9 g/L (SD) ethanol was produced from unmodified medium containing 100 g/L xylose (FIG. 18A). When fermented with the addition of KCl and KOH, a 54±5.7% (SD) increase in titer was observed, commensurate with the complete assimilation of xylose (FIG. 19). Thus, K⁺ supplementation and acidity reduction enhance tolerance in a manner impartial to the type of substrate.

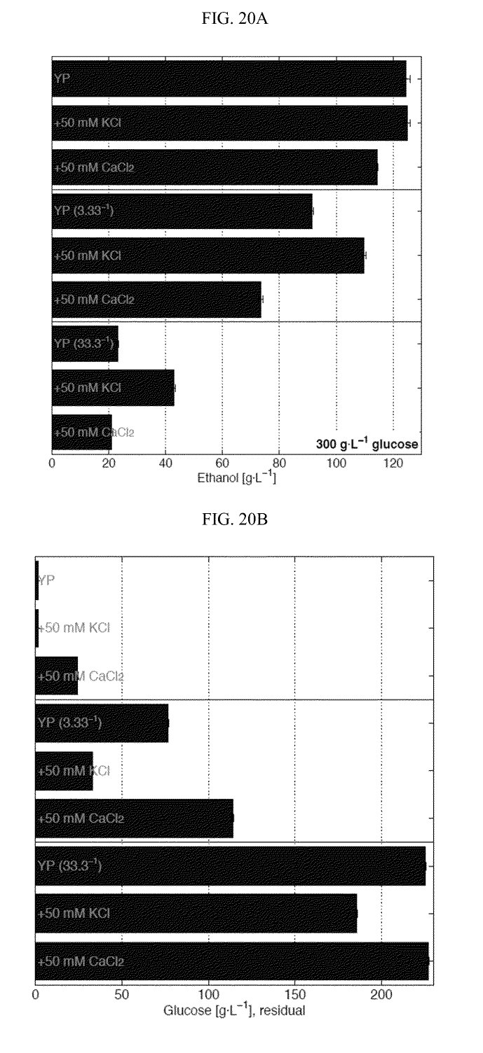

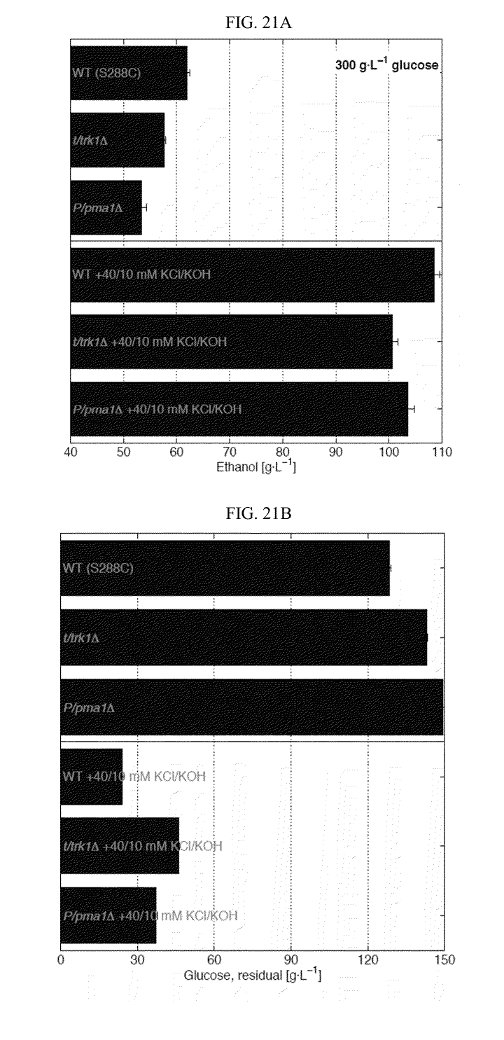

The improvements conferred by elevated K⁺ and pH generalize beyond synthetic media to chemically undefined broths, provided that such formulations do not already saturate for these effects. For example, in yeast extract-peptone (YP) medium (˜pH 6 and unknown concentrations of individual nutrients), cells ferment all sugar such that no margin is available for improvement (FIG. 20B). However, the impact of specific supplements can be assessed if the YP components are made limiting. Indeed, when YP was decreased to 30% or 3% while maintaining the same glucose concentration, supplementation with K⁺ improved ethanol output whereas additives shown to be fermentation-neutral (from FIG. 7) did not (FIG. 20A). Using YP diluted to 20%, titers of 104±0.8 g/L (SD) were produced, while the addition of K⁺ enhanced output 17±2.5% (SD) (FIG. 18B). When pH was reduced from 6 to 3.7, production was concomitantly reduced 28±0.8% (SD). However, the subsequent addition of K⁺ compensated for this decrease, restoring titers 47±2.5% to 109±1.8 g/L (SD). Thus, in media with undefined composition, extracellular K⁺ and pH are also sufficient to quantitatively modulate ethanol performance.

To isolate the effects of KCl and KOH supplementation on tolerance from other fermentation variables (e.g., decreasing turgor pressure from glucose consumption), yeast were subjected to non-physiological step increases in ethanol concentration and quantified population fractions surviving after 80 min, a period much shorter than the length of fermentation but adequate for cell viability to be impacted. In medium containing a subsistence amount of glucose that minimizes newly produced ethanol, elevated K⁺ and pH enhanced viability in shocks up to 27% (vol/vol) when compared to cells stressed in unmodified conditions (FIG. 18C). Analogous experiments performed using high glucose (mimicking the osmotic conditions of high gravity fermentation) and heightened K—Pᵢ yielded a similar result, albeit at a lower range of ethanol concentrations (FIG. 2A). These results indicate that the impact of elevated K⁺ and reduced acidity is relatively immediate, does not require adaptation to ethanol accumulation over the course of days, and is capable of overcoming the combined stress of high sugar and ethanol.

The boost in tolerance conferred by heightened K⁺ and pH extends to higher alcohols capable of serving as unmodified substitutes for gasoline. Although at lower concentrations when compared to ethanol (reflecting their increased toxicity), we observed that viability is similarly enhanced when cells are shocked using step increases of isopropanol and isobutanol (FIGS. 18D, 18E, FIG. 2B). That the improvements are not unique to ethanol suggest that these adjustments to the medium augment a more general cellular process involved in alcohol resistance or membrane integrity.

Collectively, the results provided herein suggest a toxicity model where alcohols attack viability not at threshold concentrations that solubilize lipid bilayers, but at lower concentrations that increase permeability of the plasma membrane and dissipate the cell's ionic membrane gradients. That genetically unchanged cells can be made to tolerate higher ethanol concentrations by modulating extracellular K⁺ and pH indicates that many observed tolerance thresholds (e.g., the sub-100 g/L titers from unmodified medium) represent a physiological, rather than chemical, limit. Ethanol has been known to decrease intracellular pH in a dose-dependent fashion, demonstrating that its amphipathicity permeabilizes the plasma membrane to H⁺ (and, potentially, other ions). Furthermore, that the coupled K⁺ and H⁺ gradients comprise a dominant portion of the yeast electrical membrane potential, used to power many of the cell's exchange processes with the environment, hints that the cessation of nutrient and waste transport due to gradient dissipation may be a primary mode of cell death.

### Example 10

### Bioreactor Studies

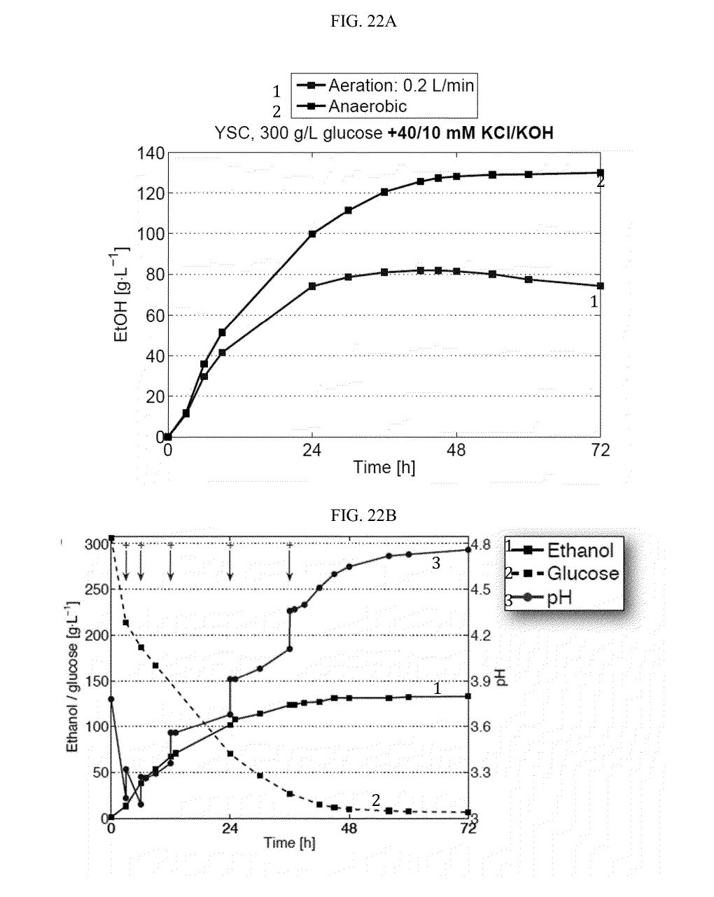

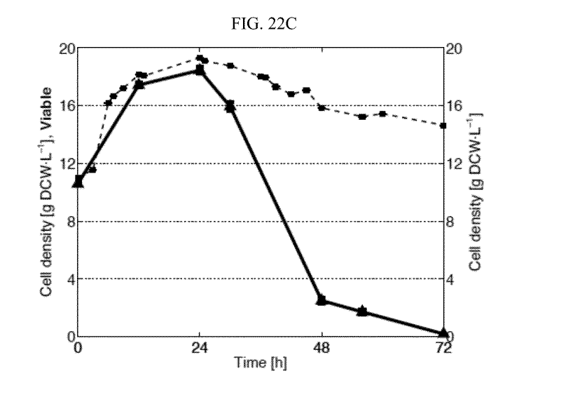

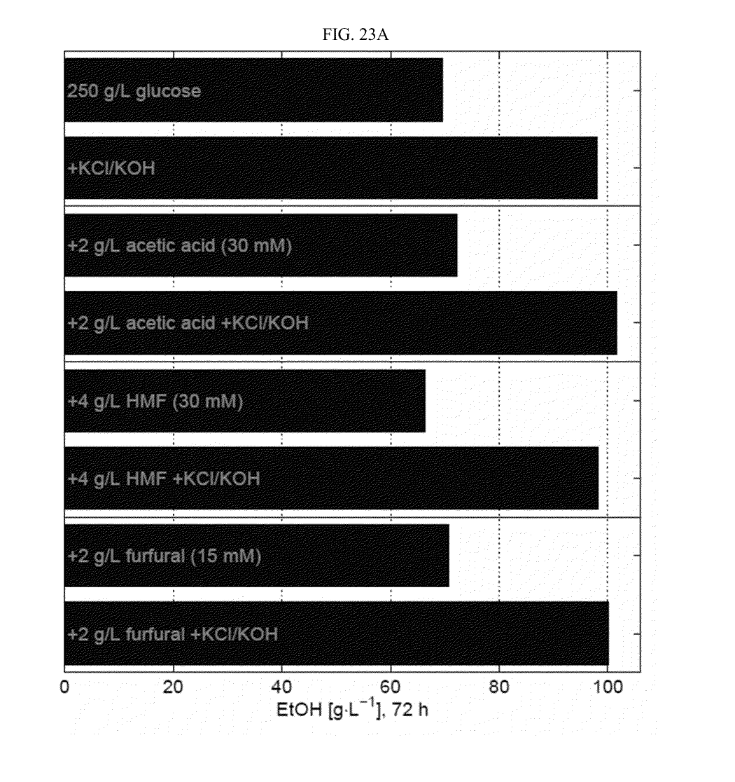

Results described above were established using shake flask or culture tube experiments. To assess whether the elevated K+ and pH enhancement methods of the present disclosure can be recapitulated in a high volume format, studies similar to those described above were conducted using bench-top bioreactors with aeration (0.2 L/min) or under anaerobic conditions (e.g., YSC, 300 g/L glucose+40/10 mM KCl/KOH for each condition). As shown in FIG. 22A, the methods provided herein do scale to industrial-like fermentation environments, producing nearly 140 g/L under anaerobic conditions.

### Example 11

### Tolerance in Cellulosic Hydrolysate

To determine whether elevated K⁺ and pH can overcome the toxicity in acid hydrolysates of cellulosic biomass, fermentation experiments were performed using synthetic lab media supplemented with the known major inhibitors (e.g., acetic acid, furfural and hydroxymethylfurfural (HMF)). At concentrations typical of those in neutralized hydrolysates, none of the inhibitors showed any inhibition when added individually; therefore, elevated K⁺ and pH enhanced production in a manner that was indistinguishable from the unsupplemented control (FIG. 23A).

All concentrations were then increased by a factor of four to 120 mM (FIG. 23B), far above what has been reported in any hydrolysate. Results demonstrate that when tested individually, elevated K⁺ and pH were still able to enhance production. Among the two most toxic inhibitor, acetic acid and furfural, the improvement was 25% and 125%, respectively. For HMF (which showed a 10% inhibition compared to the unsupplemented control), elevated K⁺ and pH appeared to completely abolish any of its toxicity: the enhanced output was similar to the supplemented control. When all three inhibitors were combined at 40 mM each, elevated K⁺ and pH enhanced production by 142%. When furfural was compared to furfuryl alcohol (its reduced equivalent) at the same toxicity, the yeast much better tolerated the alcohol. Raising K⁺ and pH then boosted production by another 54%.

Thus, the present disclosure contemplates increasing cellulosic ethanol production by converting toxic aldehydes into their equivalent alcohols and combining this process with elevated K⁺ and pH. For example, the present disclosure contemplates expressing in cells alcohol dehydrogenase enzymes that convert toxic aldehyde inhibitors, such as furfural and HMF, to their equivalent alcohols, and combining this conversion process with cellular expression of K+/H+ pumps (or K+/pH supplementation), to increase cellulosic ethanol production.

### Example 12

### Heat Tolerance

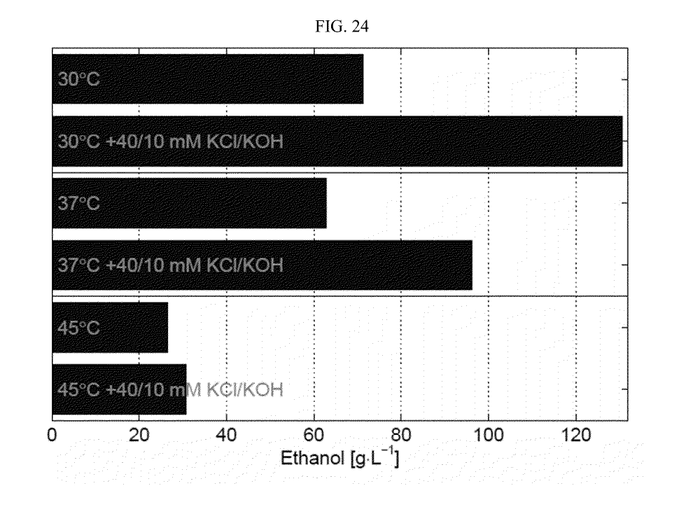

The data provided in this Example demonstrate that elevated K⁺ and pH confer heat resistance. FIG. 24 shows that the addition of KCl and KOH to fermentations enhanced fermentation at all the temperatures tested. For example, the of KCl and KOH improved ethanol production by ˜50% at 37° C. With KCl/KOH at 37° C., compared to the 30° C. time point, cells produce more ethanol, which surpasses a threshold where the 7° C. difference is now sufficient to exacerbate the membrane fluidizing effects of ethanol, leading to lower production compared with 30° C. At 45° C., KCl/KOH supplementation increases fermentation by 16% over the unmodified condition, an amount that would be of economic significance to an ethanol producer faced with cooling issues.

Without being bound by theory, heat combined with ethanol may increase permeability and, thus, dissipation of a cellular membrane's K⁺ and H⁺ ion gradients. Because KCl/KOH generally counter these fluidizing effects by increasing the K⁺/H⁺ gradients, similar improvements should be observed at any temperature with KCl/KOH supplementation or genetic enhancement of K⁺/H⁺ pumps (assuming metabolism itself hasn't yet collapsed).

### Additional Materials and Methods

### Yeast Strains.

Strains containing gene deletions in the PHO pathway were created by following a polymerase chain reaction (PCR) mediated homologous recombination technique (Longtine, M. S. et al. Yeast 14, 953-961 (1998)). In brief, primer pairs encoding the F1 and R1 plasmid-annealing sequences and sequences homologous to the 50 nucleotides directly upstream and downstream of the PHO4, PHO2, and PHM4 open reading frames were used to amplify gene deletion cassettes from the plasmid pFA6a-His3MX6. Amplification reactions were performed using the PHUSION® high-fidelity polymerase (New England Biolabs #M0530L) in 50 μl volumes containing HF buffer and thermocycled using the routine 3 step program for 35 iterations in accordance with the manufacturer's instructions. Following a lithium acetate-based protocol, 2 μg of ethanol-precipitated amplicon were transformed into 3-5 OD₆₀₀ units of strains BY4741 and BY4742 grown to mid-logarithmic phase (Gietz, R. D. et al. Yeast 11, 355-360 (1995); Brachmann, C. B. et al. Yeast 14, 115-132 (1998)).

Recombinants were recovered by histidine prototrophy, and successful targeted integration of the deletion cassette verified by PCR using a primer homologous to the promoter region of the target gene and a second primer specific to the amplicon. Validated BY4741 and BY4742 transformants containing the same gene deletion were crossed to produce the homozygous deletion strains LAMy29, LAMy30, and LAMy49.

To generate the homozygous ppz1Δ ppz2Δ double deletion, the MATa ppz1Δ and MATα ppz2Δ haploids were sourced from the Saccharomyces Genome Deletion Project collection (Life Technologies), and mated to produce the ppz1Δ::kanMX4/PPZ1 ppz2Δ::kanMX4/PPZ2 diploid. After sporulation of the heterozygote, ascospores were dissected onto YPD plates containing 200 μg/ml G418 (Sigma-Aldrich #A1720). Haploids that germinate from tetrads exhibiting a 2:2 segregation pattern unambiguously harbor the kanMX4 deletion cassette at both the PPZ1 and PPZ2 loci (Sherman, F. Meth Enzymol 350, 3-41 (2002)). The genotypes of these G418-resistant haploids were further verified by PCR using promoter- and amplicon-specific primers, and subsequently assayed for mating type via the halo test for pheromone production (using tester strains F1441 and L4564 sensitive to α- and a-factor, respectively) (Sprague, G. F. Meth Enzymol 194, 77-93 (1991)). Haploids of the opposite mating type were then crossed to produce the homozygous double deletion strain LAMy177.

To create plasmid-carrying yeast strains (e.g., LAMy96), transformation of DNA was also performed using the Gietz protocol. Typically, 500 ng of URA3-containing plasmid was introduced into 2-3 OD₆₀₀ units of cells grown to mid-logarithmic phase. Transformants were recovered through uracil prototrophy and further verified for the presence of the introduced DNA by PCR using plasmid-specific primers.

See Table 3 for a complete list of strains used in this study.

TABLE 3

| Strain | Genotype | Reference |
|---|---|---|
| BY4743 | S288C MATa/α his3Δ1/his3Δ1 leu2Δ0/leu2Δ0 LYS2/lys2Δ0 met15Δ0/MET15 | Brachmann, et al., 1998 |
| | ura3Δ0/ura3Δ0 | |
| FY4/5 | S288C MATa/α | Brachmann, et al., 1998 |
| H131-A3-AL^{CS /} | BF264-15Dau, TRP1::pTDH3-RKI1-tCYC1-pTDH3-RPE1-tCYC1, HIS2::pTDH3- | Zhou, et al., 2012 |
| F283 | TKL1-tCYC1, ADE1::pTDH3-PsTAL1-tCYC1 pUCAR1 pRS405 | |
| LAMy29 | BY4743 pho4Δ::His3MX6/pho4Δ::His3MX6 | This study |
| LAMy30 | BY4743 pho2Δ::His3MX6/pho2Δ::His3MX8 | This study |
| LAMy49 | BY4743 phm4Δ::HisMX6/phm4Δ::His3MX6 | This study |
| LAMy96 | BY4743 p416TEF | This study |
| LAMy97 | BY4743 p416TEF-PHO84 | This study |
| LAMy98 | BY4743 p416TEF-PHO90 | This study |
| LAMy123 | BY4743 p426TEF | This study |
| LAMy125 | BY4743 p426TEF-PHO84 | This study |
| LAMy126 | BY4743 p426TEF-PHO90 | This study |
| LAMy177 | BY4743 ppz1Δ::kanMX4/ppz1Δ::kanMX4 ppz2Δ::kanMX4/ppz2Δ::kanMX4 | This study |
| LAMy178 | BY4743 p416TEF-pHluorin | This study |
| LAMy184 | BY4743 p426TEF-PMA1 | This study |
| LAMy189 | LAMy177 p426TEF-PMA1 | This study |
| LAMy191 | LAMy177 p426TEF | This study |
| NCYC 479/ | Sake brewing strain, prototrophic | Akao, et al., 2011 |
| Kyokal 7 | | |
| JAY270 | Bioethanol production strain. prototrophic | Argueso, et al., 2009 |

### Plasmid Construction.

All plasmids used in this study are based on the yeast TEF1 promoted overexpression vectors (Mumberg, D. et al. Gene 156, 119-122 (1995)). To clone PHO84 and PHO90, 5′ primers encoding an NheI restriction site and 3′ primers encoding a SalI site were used to amplify either the PHO84 or PHO90 coding sequences from BY4743 genomic DNA. As above, amplification reactions were performed using the PHUSION® high-fidelity polymerase and thermocycled for 35 iterations in accordance with the manufacturer's instructions. Three μg of ethanol-precipitated PCR product were double digested with NheI-HF (New England Biolabs #R3131L) and SalI-HF (New England Biolabs #R3138L) for 2 h, and column purified using the QIAQUICK® PCR Purification Kit (QIAGEN #28106). The centromeric vector p416TEF was subjected to a sequential digest: 5 μg of plasmid was digested with XbaI (New England Biolabs #R0145L) for 1 h, the reaction heat-inactivated at 65° C. for 20 min, and adjusted by the addition of 40 mM Tris, pH 7.5 and 50 mM NaCl. The vector was further digested with SalI (New England Biolabs #R0138L) for another 1 h, and the reaction heat-inactivated for a second time at 65° C. for 20 min. Linearized p416TEF was then dephosphorylated for 1 h by alkaline phosphatase (New England Biolabs #M0290L) added directly to the digest mixture, and purified by gel extraction from 1% agarose using the QIAQUICK® Gel Extraction Kit (QIAGEN #28706). Similarly, the 2μ/high copy number plasmid p426TEF was subjected to a double digest with SpeI (New England Biolabs #R0133L) and SalI-HF for 2 h, treated immediately with alkaline phosphatase for 1 h (e.g., no heat inactivation of restriction enzymes), and purified by gel extraction from 1% agarose using the QIAQUICK® Gel Extraction Kit.

To clone PMA1, a 5′ primer encoding a SpeI restriction site and a 3′ primer encoding an XhoI site were used to amplify the PMA1 coding sequence from BY4743 genomic DNA. Approximately 3 μg of both the p426TEF vector and ethanol-precipitated PMA1 amplicon were double digested with SpeI and XhoI (New England Biolabs #R0146L) for 3 h. Linearized p426TEF was immediately dephosphorylated with alkaline phosphatase for 1 h and subsequently purified via gel extraction, while the PMA1 insert was purified using the QIAQUICK® PCR Purification Kit.

To subclone pHluorin, a 5′ primer encoding an XbaI restriction site and a 3′ primer encoding an XhoI site were used to amplify the ratiometric pHluorin coding sequence from plasmid pGM1 (gift from G. Miesenböck). Approximately 3 μg of the p416TEF plasmid and ethanol-precipitated pHluorin amplicon were double digested with XbaI (New England Biolabs #R0145L) and XhoI for 3 h. Linearized p416TEF was immediately dephosphorylated with alkaline phosphatase for 1 h and subsequently purified via gel extraction, while the pHluorin insert was purified using the QIAQUICK® PCR Purification Kit.

Ligations of inserts to end-compatible p416TEF and/or p426TEF backbones were performed using a minimum 5:1 insert:vector molar ratio in 20 μl reactions according to the manufacturer's instructions; however, twice the recommended amount of T4 DNA ligase (New England Biolabs #M0202L) was used. Reaction mixtures were transformed into chemically competent NEB 5αF′ I^{q} E. coli (New England Biolabs #C2992H), and ampicillin-resistant colonies screened for successful ligations by PCR using backbone-specific and gene-specific primers. Candidate plasmids were purified from E. coli cultures using the QIAPREP® Spin Miniprep Kit (QIAGEN #27106), and Sanger sequenced to validate the fidelity of the final product.

See Table 4 for a complete list of plasmids used in this study.

TABLE 4

| Plasmid | Insert | Reference |
|---|---|---|
| p416TEF | — | Mumberg, et al., 1995 |
| p416TEF-PHO84 | S. cerevisiae PHO84 | This study |
| p416TEF-PHO90 | S. cerevisiae PHO90 | This study |
| p416TEF-pHiuorin | Ratiometric pHluorin | This study |
| p426TEF | — | Mumberg, et al., 1995 |
| p426TEF-PHO84 | S. cerevisiae PHO84 | This study |
| p426TEF-PHO90 | S. cerevisiae PHO90 | This study |
| p426TEF-PMA1 | S. cerevisiae PMA1 | This study |

### Media and Fermentation Conditions.

To explore how ionic composition of the culture medium affects ethanol performance, chemically defined conditions were necessary which precluded, in some instances, the use of rich formulas such as yeast extract-peptone-dextrose (YEPD) medium or those containing corn steep liquor. Yeast strains were, therefore, cultured in synthetic complete medium (made from BD-Difco Yeast Nitrogen Base #233520 and remaining ingredients from Sigma-Aldrich) with nutrient drop-out for selection whenever appropriate (Sherman, F. Meth Enzymol 350, 3-41 (2002)). After the addition of a carbon source and any ionic supplements, all medium were adjusted to pH 3.8 (i.e., the equilibrium pH from the addition of 50 mM K—Pᵢ to 1×YSC), if necessary, using KOH, typically requiring <400 μM. Cultures were incubated at 30° C.: flask cultures (>25 mL) were agitated on a platform shaker at 200 RPM and smaller cultures in glass tubes on a roller drum at the maximum rotational setting. For fermentations using yeast extract-peptone (YP) medium, undiluted formulations contained the standard 10 g/L yeast extract (BD-Difco #212750) and 20 g/L peptone (BD-Difco #211830) (17), while dilutions contained these two components decreased in proportion (e.g., 2 g/L yeast extract+4 g/L peptone in the 20% dilution).

To build yeast biomass and osmotically adapt cells for high cell density and high sugar fermentations, starter cultures consisting of the unmodified base medium (i.e., 1×YSC or YSC-URA) and ˜0.3× the target fermentation sugar concentration (e.g., 100 g/L glucose) were grown until saturation, pelleted by centrifugation, and the entire cell mass used to inoculate a second “pre-fermentation” culture containing ˜0.5-0.6× the target sugar concentration (e.g., 150 g/L glucose). Pre-fermentation cultures were grown until saturation and their cell densities determined by absorbance at 600 nm of an appropriate dilution made using fresh medium. Equalized quantities of biomass (e.g., ≧200 OD₆₀₀ units) were harvested by centrifugation and re-suspended in ˜10 mL of fermentation medium to yield a cell density of OD₆₀₀≧20. In addition to the target high sugar concentration (e.g., 300 g/L glucose), the fermentation medium optionally contained the supplements under study (e.g., 50 mM K—Pᵢ) and were the first time cells were exposed to modified ionic conditions.

Fermentations to assess phenotype from genetic augmentation of the K⁺ and/or H⁺ gradients (FIG. 5B) were modified from the above as follows. To maximize expression of the hyperactivated K⁺ and H⁺ pumps, pre-fermentation cultures were grown until mid-logarithmic phase (OD₆₀₀≦3), and equalized quantities of cells (˜80 OD₆₀₀ units) harvested and re-suspended in ˜4 mL of unmodified fermentation medium to yield the target cell density of OD₆₀₀≧20.

Fermentations were conducted micro-aerobically: tube-based cultures had at least an equal volume of headspace and were capped snugly with snap-on plastic tops but not sealed with Parafilm. Samples were taken every ˜24 h over the course of 1-4 d; for simplicity, however, several figures display bar graphs of steady state or near steady state ethanol titers (e.g., FIGS. 5A-D). Typically, 16-20 μL were removed and diluted appropriately for measurement of cell density at OD₆₀₀, another 20 μL for quantification of cell viability by methylene blue staining (see below), and a final 0.5 mL pelleted and the supernatant saved for determination of ethanol concentration.

For fermentations involving pH monitoring (FIGS. 4, 5A, 12), acidity was measured directly using a Thermo Scientific Orion 2-STAR pH meter with AquaPro electrode (#9156APWP). To minimize cross contamination, the probe was immersed in HCl, pH 1 for several minutes and rinsed thoroughly with ddH₂O (double distilled via Millipore Milli-Q system) between samples. Typically, the fermentation supplemented with 50 mM K—Pᵢ was measured first to determine a target pH. Samples requiring reduction in acidity would be adjusted with KOH or NaOH (at times indicated by arrows in the figures) to match the target pH. For fermentations testing the combination of elevated KCl and pH adjustment (e.g., FIG. 4), an amount of KCl equimolar to any necessary KOH (e.g., “+KCl equiv”) was added to a separate sample to control for the impact of incremental potassium above the initial 50 mM KCl supplementation.

See Table 5 for a summary of yeast strains and conditions used in each of this study's ethanol fermentations.

TABLE 5

| Strain | Medium | Sugar (glucose/xylose) + Additive | OD_{600.0} | Display [h] |
|---|---|---|---|---|
| FY4/5 | 1x YSC | glc: 100 g/L → 150 g/L → 300 g/L ± 50 mM K—Pₜ) | ~20 | 0-96 |
| FY4/5 | 1x YSC | glc: 100 g/L → 200 g/L → 300 g/L ± 10, 25, 50, 75 mM KCl | ~20 | 0-96 |
| FY4/5 | 1x YSC | glc: 100 g/L → 150 g/L → 300 g/L ± 50 mM K—Pₜ | ~28 | 0-72 |
| | | [symbol] 300 g/L + 50 mM KCl + KOH/KCl | | |
| BY4743 | 1x YSC | glc: 100 g/L → 150 g/L → 300 g/L ± 50 mM K—Pₜ | ~28 | 48 |
| | | [symbol] 300 g/L + 50 mM KCl + KOH/KCl | | |
| LAMy49 | 1x YSC | glc: 100 g/L → 150 g/L → 300 g/L ± 50 mM K—Pₜ | ~28 | 48 |
| | | [symbol] 300 g/L + 50 mM KCl + KOH/KCl | | |
| LAMy123 | 1x YSC-URA | glc: 100 g/L → 150 g/L → 300 g/L (WT) | ~21 | 72 |
| LAMy184 | 1x YSC-URA | glc: 100 g/L → 150 g/L → 300 g/L (WT + p426TEF-PMA1) | ~20 | 72 |
| LAMy191 | 1x YSC-URA | glc: 100 g/L → 150 g/L → 300 g/L (ppz1Δppz2Δ) | ~20 | 72 |
| LAMy189 | 1x YSC-URA | glc: 100 g/L → 150 g/L → 300 g/L (ppz1Δppz2Δ + p426TEF-PMA1) | ~20 | 72 |
| NCYC 479 | 1x YSC-URA | glc: 100 g/L → 150 g/L → 300 g/L (Sake) | ~21 | 72 |
| JAY270 | 1x YSC-URA | glc: 100 g/L → 150 g/L → 300 g/L (Bioethanol) | ~20 | 72 |
| FY4/5 | 1x YSC | glc: 100 g/L → 200 g/L → 300 g/L ± 50 mM K—Pₜ | ~18 | 72 |
| BY4743 | 1x YSC | glc: 100 g/L → 200 g/L → 300 g/L ± 50 mM K—Pₜ | ~18 | 72 |
| NCYC 479 | 1x YSC | glc: 100 g/L → 200 g/L → 300 g/L ± 50 mM K—Pₜ | ~18 | 72 |
| JAY270 | 1x YSC | glc: 100 g/L → 200 g/L → 300 g/L ± 50 mM K—Pₜ | ~18 | 72 |
| H131-A3-AL^{CS} | 1x YSC | xyl: 40 g/L → 80 g/L → 100 g/L ± 50 mM K—Pₜ | ~40 | 24 |

### Ethanol Measurements.

Ethanol concentrations were determined using one of the following two methods; for consistency, however, all samples deriving from a single experiment were assayed exclusively using one method. Enzymatic quantification with the Ethanol Assay, UV-Method kit (Boehringer Mannheim/R-Biopharm #10-176-290-035) was performed according to the manufacturer's instructions on samples diluted ˜2,500⁻¹ in ddH₂O. Briefly, reactions using 1 mL of reaction mixture 2, 33.3 μL of diluted sample, and 16.7 μL of ADH (“bottle 3”) were conducted directly in polystyrene cuvettes (Bio-Rad, #223-9955), and incubated at room temperature for 5-10 mM. Absorbances of NADH at 340 nm were blanked against a reaction with ddH₂O, measured using an Ultrospec 2100 pro UV/Visible spectrophotometer (GE Healthcare Life Sciences), and normalized against the absorbance of the control solution (“bottle 4”) to determine ethanol concentrations.

Quantification by chromatography was performed on 0.5 mL of undiluted sample using an Agilent 1200 Series HPLC equipped with an Agilent 1260 Infinity Refractive Index Detector (#G1362A RID) and Aminex HPX-87H Ion Exclusion Column (Bio-Rad #125-0140). Ethanol elutes at a retention time of ˜17.3 min using 5 mM sulfuric acid at 55° C. and flow rate of 0.75 mL/min. To determine final concentrations, peak areas auto-determined by the Agilent Chemstation for LC software were interpolated off a standard curve consisting of 0-20% ethanol (by volume) prepared in 1×YSC medium.

### Viability Measurements.

To assess population viability, methylene blue (Sigma-Aldrich #M9140; a 10 mg/mL stock was prepared in ddH₂O) was added directly to aliquots of undiluted high cell density cultures to a final concentration of 1 mg/mL, and visualized immediately at 400× magnification on a Nikon Eclipse TS100 by bright field microscopy (Smart, K. A. et al. Journal of the American Society of Brewing Chemists 57, 18-23 (1999)). Images were recorded using a SPOT Insight 2 MP Firewire color camera with SPOT 5.0 software, and analyzed offline.

For each image, the number of unstained (clear) cells was quantified along with the total number (clear+stained) of cells, and the fraction of live cells determined by taking the quotient of the two. Fractions of viable cells were determined for 3-4 images per sample and used to calculate error statistics for the technical replicates (e.g., FIGS. 2A, 2B, 8A). Mean OD₆₀₀ absorbance values were multiplied by their respective mean viable fractions to arrive at the underlying viable population in OD₆₀₀ units (e.g., FIG. 1B, 3B).

All image processing and numerical analysis, including time integration of the viable populations and correlations with titer, was done in MATLAB.

### Alcohol Shock Tolerance Assay.

Over the time scale of days, the direct cellular effects of potassium supplementation and acidity reduction on fermentation are less certain as many of the variables impinging on the viable population change differentially between cultures fermented with supplementation and those without. For example, alongside higher total cell growth, K—Pᵢ supplemented cultures accumulate ethanol faster and to greater levels, potentially exacerbating toxicity; yet, they also deplete sugar faster, potentially mitigating the harm from glucose turgor stress. Although an inexact proxy of fermentation conditions, we, therefore, developed the alcohol shock tolerance assay as a means to determine viability independent of new cell growth and newly produced ethanol.

To isolate and quantify the ability of potassium supplementation and acidity reduction to increase cellular resistance to sudden changes in external alcohol concentration, cells were pre-adapted to high cell density and high sugar conditions before treatment with alcohol. For assays involving ethanol and isopropanol (FIG. 2), a starter culture of FY4/5 was grown until saturation in 1×YSC containing 100 g/L glucose, divided in half, pelleted by centrifugation, re-suspended at OD₆₀₀≈20-30 in either 1×YSC or 1×YSC+50 mM K—Pᵢ containing 300 g/L glucose, and cultured for at least 12 h. Equalized quantities of biomass (30-40 OD₆₀₀ units) were then harvested in 2 mL screw cap tubes (one per alcohol concentration), washed twice in respective fresh medium to remove fermented ethanol, and re-suspended in medium of the same composition containing 10-20% ethanol or 4-14% isopropanol. Samples were incubated at room temperature on a rotator and viability assayed after 2:15 h for ethanol, or 4 h for isopropanol, by methylene blue staining and microscopy.

For assaying tolerance to isobutanol (FIG. 5E), strain FY4/5 was cultured starting in 1×YSC containing 50 g/L glucose to build biomass, harvested and transferred to either 1× YSC or 1×YSC+50 mM KCl containing 20 g/L glucose, and grown for ˜16 h. Approximately 30 OD₆₀₀ units of the 1×YSC culture were individually harvested in 2 mL tubes, washed with 1×YSC containing 5 g/L glucose, and finally re-suspended in 1×YSC containing 5 g/L glucose and 4-6.5% isobutanol. Approximately 30 OD₆₀₀ units of the 1× YSC+50 mM KCl culture were individually harvested in 2 mL tubes, washed with either 1× YSC+50 mM KCl or 1×YSC+48 mM KCl+2 mM KOH, both containing 5 g/L glucose, and finally re-suspended in medium of the same composition containing 4-6.5% isobutanol. Samples were incubated at room temperature on a rotator and viability assayed after 1:20 h.

### Specific Productivity.

To calculate ethanol productivities per viable cell, rates of increase in ethanol titer were normalized by the average viable OD₆₀₀ during the corresponding period (FIG. 1A):

$$\frac{{EtOH}_{t} - {EtOH}_{t - 1}}{( \frac{{OD}_{600 , viable , t} + {OD}_{600 , viable , t - 1}}{2} ) ( t - {t}_{- 1} )} [ g \cdot {L}^{- 1} \cdot {OD}_{600}^{- 1} \cdot {h}^{- 1} ]$$

Intracellular pH (pHᵢ) Measurements.

To assess pHᵢ, fluorescence intensities from strains carrying a centromeric plasmid expressing ratiometric pHluorin (LAMy178) or empty vector (LAMy96) were measured in a Tecan Safire²™ microplate reader using excitation wavelengths of 395 nm and 475 nm, and common emission wavelength of 508 nm. Samples, all normalized for cell density, of 140 μL were aliquoted in duplicate to a 96-well black-walled, clear-bottom plate (Costar #3631), and the readings of the replicates averaged. Autofluorescence was removed by subtracting measurements of LAMy96 from LAMy178, both treated under identical conditions. The ratio of the intensities emitted by excitation at 395 nm to that by excitation at 475 nm is directly proportional to pHᵢ; ratios in the 0.5-1.2 range roughly correspond to pH values of ˜5.5-7 (Orij, R., et al. Microbiology (Reading, Engl) 155, 268-278 (2009)).

### Phosphate Measurements.

Concentrations of inorganic phosphate in fermentation medium (FIG. 9) were assayed colorimetrically using the Malachite Green Phosphate Assay kit (ScienCell #8118) according to the manufacturer's instructions on samples diluted 3000⁻¹.

### Anaerobic Bioreactor Fermentations.

Bioreactor fermentations were performed using a New Brunswick Scientific BioFlo 110 Bioreactor using a 1 L vessel. Dissolved oxygen (DO) and pH probes were calibrated according to the manufacturer's instructions. Cells were suspended in 500 mL (working volume) YSC medium containing 300 g/L glucose and 40 mM KCl. Anaerobic conditions are achieved within 25 min of inoculation. Continuous reading from the DO probe confirmed that anaerobicity was maintained throughout the remainder of fermentation. Manual injections totaling 10 mM KOH were added to the reactor at 3, 6, 12, 24, and 36 h using 167 μL of 6 N KOH.

### REFERENCES, EACH OF WHICH IS INCORPORATED BY REFERENCE HEREIN

1. Demain, A. L. Biosolutions to the energy problem. J Ind Microbiol Biotechnol 36, 319-332 (2009).
2. Bai, F. W., Chen, L. J., Zhang, Z., Anderson, W. A. & Moo-Young, M. Continuous ethanol production and evaluation of yeast cell lysis and viability loss under very high gravity medium conditions. J Biotechnol 110, 287-293 (2004).
3. Stambuk, B. U., Dunn, B., Alves, S. L., Duval, E. H. & Sherlock, G. Industrial fuel ethanol yeasts contain adaptive copy number changes in genes involved in vitamin B1 and B6 biosynthesis. Genome Res 19, 2271-2278 (2009).
4. van Voorst, F., Houghton-Larsen, J., Jønson, L., Kielland-Brandt, M. C. & Brandt, A. Genome-wide identification of genes required for growth of Saccharomyces cerevisiae under ethanol stress. Yeast 23, 351-359 (2006).
5. Swinnen, S. et al. Identification of novel causative genes determining the complex trait of high ethanol tolerance in yeast using pooled-segregant whole-genome sequence analysis. Genome Res 22, 975-984 (2012).
6. Stanley, D., Bandara, A., Fraser, S., Chambers, P. J. & Stanley, G. A. The ethanol stress response and ethanol tolerance of Saccharomyces cerevisiae. J Appl Microbiol 109, 13-24 (2010).
7. Alper, H., Moxley, J., Nevoigt, E., Fink, G. R. & Stephanopoulos, G. Engineering yeast transcription machinery for improved ethanol tolerance and production. Science 314, 1565-1568 (2006).
8. Madeira, A. et al. Effect of ethanol on fluxes of water and protons across the plasma membrane of Saccharomyces cerevisiae. FEMS Yeast Res 10, 252-258 (2010).
9. Dickey, A. N. & Faller, R. How alcohol chain-length and concentration modulate hydrogen bond formation in a lipid bilayer. Biophys J 92, 2366-2376 (2007).
10. Chanda, J. & Bandyopadhyay, S. Perturbation of phospholipid bilayer properties by ethanol at a high concentration. Langmuir 22, 3775-3781 (2006).
11. Lewis, J. A., Elkon, I. M., McGee, M. A., Higbee, A. J. & Gasch, A. P. Exploiting natural variation in Saccharomyces cerevisiae to identify genes for increased ethanol resistance. Genetics 186, 1197-1205 (2010).
12. Argueso, J. L. et al. Genome structure of a Saccharomyces cerevisiae strain widely used in bioethanol production. Genome Res 19, 2258-2270 (2009).
13. O'Rourke, S. M. & Herskowitz, I. Unique and redundant roles for HOG MAPK pathway components as revealed by whole-genome expression analysis. Mol Biol Cell 15, 532-542 (2004).
14. Kim, N.-R. et al. Mutations of the TATA-binding protein confer enhanced tolerance to hyperosmotic stress in Saccharomyces cerevisiae. Appl Microbiol Biotechnol (2013). doi:10.1007/s00253-013-4985-8
15. Lam, F. H., Steger, D. J. & O'Shea, E. K. Chromatin decouples promoter threshold from dynamic range. Nature 453, 246-250 (2008).
16. Wykoff, D. D. & O'Shea, E. K. Phosphate transport and sensing in Saccharomyces cerevisiae. Genetics 159, 1491-1499 (2001).
17. Serrano, R., Kielland-Brandt, M. C. & Fink, G. R. Yeast plasma membrane ATPase is essential for growth and has homology with (Na++K+), K+- and Ca2+-ATPases. Nature 319, 689-693 (1986).
18. Gaber, R. F., Styles, C. A. & Fink, G. R. TRK1 encodes a plasma membrane protein required for high-affinity potassium transport in Saccharomyces cerevisiae. Mol Cell Biol 8, 2848-2859 (1988).
19. Cohen, A., Perzov, N., Nelson, H. & Nelson, N. A novel family of yeast chaperons involved in the distribution of V-ATPase and other membrane proteins. J Biol Chem 274, 26885-26893 (1999).
20. Yenush, L., Merchan, S., Holmes, J. & Serrano, R. pH-Responsive, posttranslational regulation of the Trk1 potassium transporter by the type 1-related Ppz1 phosphatase. Mol Cell Biol 25, 8683-8692 (2005).
21. Yenush, L., Mulet, J. M., Ariño, J. & Serrano, R. The Ppz protein phosphatases are key regulators of K+ and pH homeostasis: implications for salt tolerance, cell wall integrity and cell cycle progression. EMBO J 21, 920-929 (2002).
22. Katou, T., Namise, M., Kitagaki, H., Akao, T. & Shimoi, H. QTL mapping of sake brewing characteristics of yeast. J. Biosci. Bioeng. 107, 383-393 (2009).
23. Somerville, C. Biofuels. Curr Biol 17, R115-9 (2007).
24. Zhou, H., Cheng, J.-S., Wang, B. L., Fink, G. R. & Stephanopoulos, G. Xylose isomerase overexpression along with engineering of the pentose phosphate pathway and evolutionary engineering enable rapid xylose utilization and ethanol production by Saccharomyces cerevisiae. Metab Eng 14, 611-622 (2012).
25. Gjersing, E., Happs, R. M., Sykes, R. W., Doeppke, C. & Davis, M. F. Rapid determination of sugar content in biomass hydrolysates using nuclear magnetic resonance spectroscopy. Biotechnol Bioeng 110, 721-728 (2013).
26. Atsumi, S., Hanai, T. & Liao, J. C. Non-fermentative pathways for synthesis of branched-chain higher alcohols as biofuels. Nature 451, 86-89 (2008).
27. Avalos, J. L., Fink, G. R. & Stephanopoulos, G. Compartmentalization of metabolic pathways in yeast mitochondria improves the production of branched-chain alcohols. Nat Biotechnol 31, 335-341 (2013).
28. Atsumi, S. et al. Evolution, genomic analysis, and reconstruction of isobutanol tolerance in Escherichia coli. Mol Syst Biol 6, 449 (2010).
29. Madrid, R., Gómez, M. J., Ramos, J. & Rodríguez-Navarro, A. Ectopic potassium uptake in trk1 trk2 mutants of Saccharomyces cerevisiae correlates with a highly hyperpolarized membrane potential. J Biol Chem 273, 14838-14844 (1998).
30. Orij, R., Brul, S. & Smits, G. J. Intracellular pH is a tightly controlled signal in yeast. Biochim Biophys Acta 1810, 933-944 (2011).
31. Sá-Correia, I., Santos, dos, S. C., Teixeira, M. C., Cabrito, T. R. & Mira, N. P. Drug:H+ antiporters in chemical stress response in yeast. Trends Microbiol. 17, 22-31 (2009).
32. Cyert, M. S. & Philpott, C. C. Regulation of Cation Balance in Saccharomyces cerevisiae. Genetics 193, 677-713 (2013).
33. Horák, J. Yeast nutrient transporters. Biochim Biophys Acta 1331, 41-79 (1997).
34. Miesenböck, G., De Angelis, D. A. & Rothman, J. E. Visualizing secretion and synaptic transmission with pH-sensitive green fluorescent proteins. Nature 394, 192-195 (1998).

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

It should also be understood that, unless clearly indicated to the contrary, in any methods claimed herein that include more than one step or act, the order of the steps or acts of the method is not necessarily limited to the order in which the steps or acts of the method are recited.

All references (e.g., published journal articles, books, etc.), patents and patent applications disclosed herein are incorporated by reference with respect to the subject matter for which each is cited, which, in some cases, may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of alcohol production, comprising culturing yeast cells in culture medium that comprises fermentable feedstock and a potassium salt, wherein the potassium salt is in an amount sufficient to produce at least 100 g/L alcohol.

2. The method of claim 1, wherein the potassium salt is potassium phosphate monobasic (KH₂PO₄), potassium phosphate dibasic (K₂HPO₄), potassium sulfate (K₂SO₄) or potassium chloride (KCl).

3. The method of claim 1, wherein the potassium salt is in an amount sufficient to produce at least 130 g/L alcohol, at least 140 g/L alcohol, or at least 150 g/L alcohol.

4-5. (canceled)

6. The method of claim 1, wherein the alcohol is ethanol, isopropanol or isobutanol.

7. The method of claim 1, wherein the potassium salt is KH₂PO₄, or wherein the potassium salt is KCl and the culture medium further comprises potassium hydroxide (KOH).

8. (canceled)

9. The method of claim 7, wherein the KOH is in an amount sufficient to maintain, in the culture medium, a pH of at least 3.5.

10. The method of claim 1, wherein the concentration of potassium salt is about 25 mM to about 100 mM.

11. (canceled)

12. The method of claim 1, wherein the fermentable feedstock comprises cellulosic feedstock or fermentable sugar.

13. (canceled)

14. The method of claim 12, wherein the fermentable feedstock comprises a fermentable sugar selected from glucose and xylose.

15. (canceled)

16. The method of claim 12, wherein the concentration of the fermentable sugar is about 50 g/L to about 400 g/L.

17-18. (canceled)

19. The method of claim 12, wherein the yeast cells are Saccharomyces cerevisiae cells.

20. The method of claim 12, wherein the yeast cells are industrial yeast cells.

21. The method of claim 12, wherein the yeast cells are NCYC 479 (Sake) yeast cells, PE-2 (Bioethanol) cells, or ETHANOL RED® cells.

22-23. (canceled)

24. The method of claim 1, wherein the yeast cells have been previously modified to produce ethanol.

25. The method of claim 1, wherein the yeast cells express a cellulase and/or a hemicellulase.

26. The method of claim 1, wherein the yeast cells are engineered to comprise a modified potassium transport gene encoding a polypeptide that increases cellular influx of potassium relative to an unmodified yeast cell and a modified proton transport gene encoding a polypeptide that increases the cellular efflux of protons relative to an unmodified yeast cell.

27. The method of claim 26, wherein at least 160 g/L or at least 170 g/L of alcohol is produced.

28. (canceled)

29. The method of claim 1, wherein the yeast cells are engineered to express an enzyme that converts aldehydes into their equivalent alcohols.

30. The method of claim 29, wherein the enzyme is selected from the group consisting of: alcohol dehydrogenases, aldehyde dehydrogenases, aldehyde reductases, oxidative stress activators, catalases activated by YAP1, xylose reductases, and methylglyoxal reductases.

31. The method of claim 30, wherein the enzyme is an alcohol dehydrogenase, an aldehyde dehydrogenase or an aldehyde reductase.

32. The method of claim 31, wherein the enzyme is an alcohol dehydrogenase selected from ADH1, ADH2, ADH6, ADH7 and SFA1, or an aldehyde dehydrogenase selected from ALD4 and ALD5, or an aldehyde reductase selected from GRE3 and ARI1.

33-36. (canceled)

37. The method of claim 1, wherein the yeast cells are cultured at a temperature of higher than 30° C.

38. (canceled)

39. A composition comprising yeast in culture medium that comprises fermentable feedstock and a potassium salt, wherein the potassium salt is in an amount sufficient to produce at least 100 g/L alcohol.

40-74. (canceled)

75. An alcohol tolerant yeast cell engineered to comprise a modified potassium transport gene encoding a polypeptide that increases cellular influx of potassium relative to an unmodified yeast cell and a modified proton transport gene encoding a polypeptide that increases the cellular efflux of protons relative to an unmodified yeast cell.

76-115. (canceled)

116. A method of producing the alcohol tolerant yeast cell of claim 75, the method comprising modifying in a yeast cell a potassium transport gene and a proton transport gene, thereby producing an alcohol tolerant yeast cell with an increased cellular influx of potassium and an increased cellular efflux of protons relative to an unmodified yeast cell.

117-156. (canceled)
